SEQUENCE LISTING (56 sequences)

SEQ ID NO: 1 - protein, 25 aa, Artificial Sequence
  4811 HFR1
QVQLVESGGGLVQAGGSLRLSCAAS

SEQ ID NO: 2 - protein, 8 aa, Artificial Sequence
  4811 HCDR1;hu4811 HCDR1;hu4811FGLF HCDR1
GHTPSNYA

SEQ ID NO: 3 - protein, 17 aa, Artificial Sequence
  4811 HFR2;hu4811 HFR2
MGWFRQTPGKEREFVAA

SEQ ID NO: 4 - protein, 13 aa, Artificial Sequence
  4811 HCDR2;hu4811 HCDR2;hu4811FGLF HCDR2
ITWSAVTRDDAIP

SEQ ID NO: 5 - protein, 38 aa, Artificial Sequence
  4811 HFR3
YYRTSVKGRFTISRDNAKNTVYLQMNSLKPEDTAVYYC

SEQ ID NO: 6 - protein, 14 aa, Artificial Sequence
  4811 HCDR3;hu4811 HCDR3;hu4811FGLF HCDR3
AVDASLVMTPTPRY

SEQ ID NO: 7 - protein, 11 aa, Artificial Sequence
  4811 HFR4
WGQGTQVTVSS

SEQ ID NO: 8 - protein, 25 aa, Artificial Sequence
  D1 HFR1
QVQLVESGGGLVQAGGSLRLSCVAS

SEQ ID NO: 9 - protein, 8 aa, Artificial Sequence
  D1 HCDR1;huD1 HCDR1;huD1FGLF HCDR1
GRTSTTSV

SEQ ID NO: 10 - protein, 17 aa, Artificial Sequence
  D1 HFR2;huD1 HFR2
MGWFRQAPGKERRFVAA

SEQ ID NO: 11 - protein, 13 aa, Artificial Sequence
  D1 HCDR2;huD1 HCDR2;huD1FGLF HCDR2
ITRTDDRQNDGIT

SEQ ID NO: 12 - protein, 38 aa, Artificial Sequence
  D1 HFR3
YYGESVKGRFTISRDNDKNTVYLQMNSLKPEDTAVYYC

SEQ ID NO: 13 - protein, 14 aa, Artificial Sequence
  D1 HCDR3;huD1 HCDR3;huD1FGLF HCDR3
AAHTSLVFTPDPRY

SEQ ID NO: 14 - protein, 11 aa, Artificial Sequence
  D1 HFR4
WGQGTPVTVPS

SEQ ID NO: 15 - protein, 126 aa, Artificial Sequence
  Single-domain antibody 4811 (Heavy chain variable domain)
QVQLVESGGGLVQAGGSLRLSCAASGHTPSNYAMGWFRQTPGKEREFVAAITWSAVTRDDAIPYYRTSVKGRFTISRDNAKNTVYLQMNSLKPEDTAVYYCAVDASLVMTPTPRYWGQGTQVTVSS

SEQ ID NO: 16 - protein, 126 aa, Artificial Sequence
  Single-domain antibody D1 (Heavy chain variable domain)
QVQLVESGGGLVQAGGSLRLSCVASGRTSTTSVMGWFRQAPGKERRFVAAITRTDDRQNDGITYYGESVKGRFTISRDNDKNTVYLQMNSLKPEDTAVYYCAAHTSLVFTPDPRYWGQGTPVTVPS

SEQ ID NO: 17 - protein, 126 aa, Artificial Sequence
  Single-domain antibody C6 (Heavy chain variable domain)
QVQLVESGGGLVETGSSLRLSCKSSGHSFSDYSLAWFRQAPGKEREFVAAITWSDVPRDDAVTYYRVDVKGRFTISRDNAKNTVYLQMNSLKPEDTAVYYCAVDASLVMTLTPRYWGQGTQVTVSS

SEQ ID NO: 18 - protein, 126 aa, Artificial Sequence
  Single-domain antibody C2 (Heavy chain variable domain)
QVQLVESGGGLVQAGGSLRLSCVASGLTFSNYAMGWFRQGPGKDRDFVAAITRSDERQNDGVTYYRVSVKGRFTISRDNANDTVYLQMNSLKPEDTAIYYCAADPSLVFSPTLRYWGQGTLVTVSS

SEQ ID NO: 19 - protein, 126 aa, Artificial Sequence
  Single-domain antibody D10 (Heavy chain variable domain)
QVQLVESGGGLVQPGGSLRLSCAASGFSLNNYVIGWFRQAPGKDRDFVAAITRSDERQNDGVTYYRVSVKGRFTISRDNANDTVYLQMNSLKPEDTAIYYCAADPSLVFSPTPRYWGQGTLVTVSS

SEQ ID NO: 20 - protein, 126 aa, Artificial Sequence
  Single-domain antibody huD1 (Heavy chain variable domain)
QVQLVESGGGLVQPGGSLRLSCAASGRTSTTSVMGWFRQAPGKERRFVAAITRTDDRQNDGITYYADSVKGRFTISRDNSKNTLYLQMNSLRAEDTAVYYCAAHTSLVFTPDPRYWGQGTLVTVSS

SEQ ID NO: 21 - protein, 126 aa, Artificial Sequence
  Single-domain antibody huD1FGLF (Heavy chain variable domain)
QVQLVESGGGLVQPGGSLRLSCAASGRTSTTSVMGWFRQAPGKGLEFVAAITRTDDRQNDGITYYADSVKGRFTISRDNSKNTLYLQMNSLRAEDTAVYYCAAHTSLVFTPDPRYWGQGTLVTVSS

SEQ ID NO: 22 - protein, 126 aa, Artificial Sequence
  Single-domain antibody hu4811 (Heavy chain variable domain)
QVQLVESGGGLVQPGGSLRLSCAASGHTPSNYAMGWFRQTPGKEREFVAAITWSAVTRDDAIPYYADSVKGRFTISRDNSKNTLYLQMNSLRAEDTAVYYCAVDASLVMTPTPRYWGQGTLVTVSS

SEQ ID NO: 23 - protein, 126 aa, Artificial Sequence
  Single-domain antibody hu4811FGLF (Heavy chain variable domain)
QVQLVESGGGLVQPGGSLRLSCAASGHTPSNYAMGWFRQAPGKGLEFVAAITWSAVTRDDAIPYYADSVKGRFTISRDNSKNTLYLQMNSLRAEDTAVYYCAVDASLVMTPTPRYWGQGTLVTVSS

SEQ ID NO: 24 - DNA, 378 bp, Artificial Sequence
  Single-domain antibody 4811 (Heavy chain variable domain) Nucleic acid sequence
caggtgcagctggtggagtctgggggaggattggtgcaggctgggggctctctgagactctcctgtgcagcctctggacacacccccagtaattatgccatgggctggttccgccagactccagggaaggagcgtgagttcgtagcagctattacctggagtgctgttacccgggatgatgcaataccatactatagaacctccgtgaagggccgattcaccatctcccgagacaacgccaagaacacggtgtatctgcaaatgaacagcctgaaacctgaggacacggccgtttattactgtgcagtagacgcttcattggtcatgacgcctacaccgcgctactggggccaggggacccaggtcaccgtctcgtca

SEQ ID NO: 25 - DNA, 378 bp, Artificial Sequence
  Single-domain antibody D1 (Heavy chain variable domain) Nucleic acid sequence
caggtgcagctggtggagtctgggggaggattggtgcaggctgggggctctctgagactctcctgtgtggcgtctggacgtacctcaacgacgtctgtcatgggctggttccgccaggcgccagggaaggagcgtaggtttgtagcagctattacccgtactgatgatcgccagaatgatggtataacatactatggagagtccgtgaagggccgattcaccatctcccgagacaacgacaagaacacggtgtatctgcaaatgaacagcctgaaacctgaggacacggccgtttattactgtgcagcacacacttcgttggtatttacgcctgacccgcgctactggggccaggggaccccggtcaccgtcccctca

SEQ ID NO: 26 - DNA, 378 bp, Artificial Sequence
  Single-domain antibody huD1 (Heavy chain variable domain) Nucleic acid sequence
caggtgcagctggtggagtctgggggaggcttggtacagcctggggggtccctgagactctcctgtgcagcctctggacgtacctcaacgacgtctgtcatgggctggttccgccaggcgccagggaaggagcgtaggtttgtagcagctattacccgtactgatgatcgccagaatgatggtataacatactacgcagactccgtgaagggccggttcaccatctccagagacaattccaagaacacgctgtatctgcaaatgaacagcctgagagccgaggacacggccgtatattactgtgcagcacacacttcgttggtatttacgcctgacccgcgctactggggccagggaaccctggtcaccgtctcctca

SEQ ID NO: 27 - DNA, 378 bp, Artificial Sequence
  Single-domain antibody huD1FGLF (Heavy chain variable domain)Nucleic acid sequence
caggtgcagctggtggagtctgggggaggcttggtacagcctggggggtccctgagactctcctgtgcagcctctggacgtacctcaacgacgtctgtcatgggctggttccgccaggctccagggaaggggctggagtttgtcgcagctattacccgtactgatgatcgccagaatgatggtataacatactacgcagactccgtgaagggccggttcaccatctccagagacaattccaagaacacgctgtatctgcaaatgaacagcctgagagccgaggacacggccgtatattactgtgcagcacacacttcgttggtatttacgcctgacccgcgctactggggccagggaaccctggtcaccgtctcctca

SEQ ID NO: 28 - DNA, 378 bp, Artificial Sequence
  Single-domain antibody hu4811 (Heavy chain variable domain)Nucleic acid sequence
caggtgcagctggtggagtctgggggaggcttggtacagcctggggggtccctgagactctcctgtgcagcctctggacacacccccagtaattatgccatgggctggttccgccagactccagggaaggagcgtgagttcgtagcagctattacctggagtgctgttacccgggatgatgcaataccatactacgcagactccgtgaagggccggttcaccatctccagagacaattccaagaacacgctgtatctgcaaatgaacagcctgagagccgaggacacggccgtatattactgtgcagtagacgcttcattggtcatgacgcctacaccgcgctactggggccagggaaccctggtcaccgtctcctca

SEQ ID NO: 29 - DNA, 378 bp, Artificial Sequence
  Single-domain antibody hu4811FGLF (Heavy chain variable domain) Nucleic acid sequence
caggtgcagctggtggagtctgggggaggcttggtacagcctggggggtccctgagactctcctgtgcagcctctggacacacccccagtaattatgccatgggctggttccgccaggctccagggaaggggctggagttcgtcgcagctattacctggagtgctgttacccgggatgatgcaataccatactacgcagactccgtgaagggccggttcaccatctccagagacaattccaagaacacgctgtatctgcaaatgaacagcctgagagccgaggacacggccgtatattactgtgcagtagacgcttcattggtcatgacgcctacaccgcgctactggggccagggaaccctggtcaccgtctcctca

SEQ ID NO: 30 - protein, 25 aa, Artificial Sequence
  hu4811 HFR1;hu4811FGLF HFR1;huD1 HFR1;huD1FGLF HFR1
QVQLVESGGGLVQPGGSLRLSCAAS

SEQ ID NO: 31 - protein, 38 aa, Artificial Sequence
  hu4811 HFR3;hu4811FGLF HFR3;huD1 HFR3;huD1FGLF HFR3
YYADSVKGRFTISRDNSKNTLYLQMNSLRAEDTAVYYC

SEQ ID NO: 32 - protein, 11 aa, Artificial Sequence
  hu4811 HFR4;hu4811FGLF HFR4;huD1 HFR4;huD1FGLF HFR4
WGQGTLVTVSS

SEQ ID NO: 33 - protein, 17 aa, Artificial Sequence
  hu4811FGLF HFR2;huD1FGLF HFR2
MGWFRQAPGKGLEFVAA

SEQ ID NO: 34 - DNA, 75 bp, Artificial Sequence
  hu4811 HFR1 Nucleic acid sequence;hu4811FGLF HFR1 Nucleic acid sequence;huD1 HFR1 Nucleic acid sequence;huD1FGLF HFR1 Nucleic acid sequence
caggtgcagctggtggagtctgggggaggcttggtacagcctggggggtccctgagactctcctgtgcagcctct

SEQ ID NO: 35 - DNA, 24 bp, Artificial Sequence
  hu4811 HCDR1 Nucleic acid sequence;hu4811FGLF HCDR1 Nucleic acid sequence
ggacacacccccagtaattatgcc

SEQ ID NO: 36 - DNA, 51 bp, Artificial Sequence
  hu4811 HFR2 Nucleic acid sequence
atgggctggttccgccagactccagggaaggagcgtgagttcgtagcagct

SEQ ID NO: 37 - DNA, 39 bp, Artificial Sequence
  hu4811 HCDR2 Nucleic acid sequence;hu4811FGLF HCDR2 Nucleic acid sequence
attacctggagtgctgttacccgggatgatgcaatacca

SEQ ID NO: 38 - DNA, 114 bp, Artificial Sequence
  hu4811 HFR3 Nucleic acid sequence;hu4811FGLF HFR3 Nucleic acid sequence;huD1 HFR3 Nucleic acid sequence;huD1FGLF HFR3 Nucleic acid sequence
tactacgcagactccgtgaagggccggttcaccatctccagagacaattccaagaacacgctgtatctgcaaatgaacagcctgagagccgaggacacggccgtatattactgt

SEQ ID NO: 39 - DNA, 42 bp, Artificial Sequence
  hu4811 HCDR3 Nucleic acid sequence;hu4811FGLF HCDR3 Nucleic acid sequence
gcagtagacgcttcattggtcatgacgcctacaccgcgctac

SEQ ID NO: 40 - DNA, 33 bp, Artificial Sequence
  hu4811 HFR4 Nucleic acid sequence;hu4811FGLF HFR4 Nucleic acid sequence;huD1 HFR4 Nucleic acid sequence;huD1FGLF HFR4 Nucleic acid sequence
tggggccagggaaccctggtcaccgtctcctca

SEQ ID NO: 41 - DNA, 51 bp, Artificial Sequence
  hu4811FGLF HFR2 Nucleic acid sequence
atgggctggttccgccaggctccagggaaggggctggagttcgtcgcagct

SEQ ID NO: 42 - DNA, 24 bp, Artificial Sequence
  huD1 HCDR1 Nucleic acid sequence;huD1FGLF HCDR1 Nucleic acid sequence
ggacgtacctcaacgacgtctgtc

SEQ ID NO: 43 - DNA, 51 bp, Artificial Sequence
  huD1 HFR2 Nucleic acid sequence
atgggctggttccgccaggcgccagggaaggagcgtaggtttgtagcagct

SEQ ID NO: 44 - DNA, 39 bp, Artificial Sequence
  huD1 HCDR2 Nucleic acid sequence;huD1FGLF HCDR2 Nucleic acid sequence
attacccgtactgatgatcgccagaatgatggtataaca

SEQ ID NO: 45 - DNA, 42 bp, Artificial Sequence
  huD1 HCDR3 Nucleic acid sequence;huD1FGLF HCDR3 Nucleic acid sequence
gcagcacacacttcgttggtatttacgcctgacccgcgctac

SEQ ID NO: 46 - DNA, 51 bp, Artificial Sequence
  huD1FGLF HFR2 Nucleic acid sequence
atgggctggttccgccaggctccagggaaggggctggagtttgtcgcagct

SEQ ID NO: 47 - protein, 126 aa, Artificial Sequence
  4811/hu4811/hu4811FGLFSingle-domain antibody general formula
  misc_feature (14)..(14): Xaa = Ala or Pro
  misc_feature (40)..(40): Xaa = Ala or Thr
  misc_feature (44)..(44): Xaa = Glu or Gly
  misc_feature (45)..(45): Xaa = Leu or Arg
  misc_feature (66)..(66): Xaa = Ala or Arg
  misc_feature (67)..(67): Xaa = Asp or Thr
  misc_feature (80)..(80): Xaa = Ala or Ser
  misc_feature (84)..(84): Xaa = Leu or Val
  misc_feature (92)..(92): Xaa = Lys or Arg
  misc_feature (93)..(93): Xaa = Ala or Pro
  misc_feature (121)..(121): Xaa = Leu or Gln
QVQLVESGGGLVQXGGSLRLSCAASGHTPSNYAMGWFRQXPGKXXEFVAAITWSAVTRDDAIPYYXXSVKGRFTISRDNXKNTXYLQMNSLXXEDTAVYYCAVDASLVMTPTPRYWGQGTXVTVSS

SEQ ID NO: 48 - protein, 126 aa, Artificial Sequence
  D1/huD1/huD1FGLFSingle-domain antibody general formula
  misc_feature (14)..(14): Xaa = Ala or Pro
  misc_feature (23)..(23): Xaa = Ala or Val
  misc_feature (44)..(44): Xaa = Glu or Gly
  misc_feature (45)..(45): Xaa = Leu or Arg
  misc_feature (46)..(46): Xaa = Glu or Arg
  misc_feature (66)..(66): Xaa = Ala or Gly
  misc_feature (67)..(67): Xaa = Asp or Glu
  misc_feature (80)..(80): Xaa = Asp or Ser
  misc_feature (84)..(84): Xaa = Leu or Val
  misc_feature (92)..(92): Xaa = Lys or Arg
  misc_feature (93)..(93): Xaa = Ala or Pro
  misc_feature (121)..(121): Xaa = Leu or Pro
  misc_feature (125)..(125): Xaa = Pro or Ser
QVQLVESGGGLVQXGGSLRLSCXASGRTSTTSVMGWFRQAPGKXXXFVAAITRTDDRQNDGITYYXXSVKGRFTISRDNXKNTXYLQMNSLXXEDTAVYYCAAHTSLVFTPDPRYWGQGTXVTVXS

SEQ ID NO: 49 - protein, 25 aa, Artificial Sequence
  4811/hu4811/hu4811FGLF HFR1general formula
  misc_feature (14)..(14): Xaa = Ala or Pro
QVQLVESGGGLVQXGGSLRLSCAAS

SEQ ID NO: 50 - protein, 25 aa, Artificial Sequence
  D1/huD1/huD1FGLF HFR1 general formula
  misc_feature (14)..(14): Xaa = Ala or Pro
  misc_feature (23)..(23): Xaa = Ala or Val
QVQLVESGGGLVQXGGSLRLSCXAS

SEQ ID NO: 51 - protein, 17 aa, Artificial Sequence
  4811/hu4811/hu4811FGLF HFR2 general formula
  misc_feature (7)..(7): Xaa = Ala or Thr
  misc_feature (11)..(11): Xaa = Glu or Gly
  misc_feature (12)..(12): Xaa = Leu or Arg
MGWFRQXPGKXXEFVAA

SEQ ID NO: 52 - protein, 17 aa, Artificial Sequence
  D1/huD1/huD1FGLF HFR2 general formula
  misc_feature (11)..(11): Xaa = Glu or Gly
  misc_feature (12)..(12): Xaa = Leu or Arg
  misc_feature (13)..(13): Xaa = Glu or Arg
MGWFRQAPGKXXXFVAA

SEQ ID NO: 53 - protein, 38 aa, Artificial Sequence
  4811/hu4811/hu4811FGLF HFR3 general formula
  misc_feature (3)..(3): Xaa = Ala or Arg
  misc_feature (4)..(4): Xaa = Asp or Thr
  misc_feature (17)..(17): Xaa = Ala or Ser
  misc_feature (21)..(21): Xaa = Leu or Val
  misc_feature (29)..(29): Xaa = Lys or Arg
  misc_feature (30)..(30): Xaa = Ala or Pro
YYXXSVKGRFTISRDNXKNTXYLQMNSLXXEDTAVYYC

SEQ ID NO: 54 - protein, 38 aa, Artificial Sequence; identical to SEQ ID NO: 53
  D1/huD1/huD1FGLF HFR3 general formula
  misc_feature (3)..(3): Xaa = Ala or Gly
  misc_feature (4)..(4): Xaa = Asp or Glu
  misc_feature (17)..(17): Xaa = Asp or Ser
  misc_feature (21)..(21): Xaa = Leu or Val
  misc_feature (29)..(29): Xaa = Lys or Arg
  misc_feature (30)..(30): Xaa = Ala or Pro

SEQ ID NO: 55 - protein, 11 aa, Artificial Sequence
  4811/hu4811/hu4811FGLF HFR4 general formula
  misc_feature (6)..(6): Xaa = Leu or Gln
WGQGTXVTVSS

SEQ ID NO: 56 - protein, 11 aa, Artificial Sequence
  D1/huD1/huD1FGLF HFR4 general formula
  misc_feature (6)..(6): Xaa = Leu or Pro
  misc_feature (10)..(10): Xaa = Pro or Ser
WGQGTXVTVXS

(19) United States
(12) Patent Application Publication
(10) Pub. No.: US 2025/0171550 A1
(43) Pub. Date: May 29, 2025

(54) BCMA-TARGETING SINGLE-DOMAIN ANTIBODY

(71) Applicant: NINGBO T-MAXIMUM BIOPHARMACEUTICALS CO., LTD., Zhejiang (CN)

(72) Inventors: Xiaoyun SHANG, Jiangsu (CN); Jialu LI, Jiangsu (CN); Shaowen MA, Jiangsu (CN)

(21) Appl. No.: 18/270,104

(22) PCT Filed: Dec. 28, 2021
(86) PCT No.: PCT/CN2021/141930

§ 371 Date: Jun. 28, 2023

(30) Foreign Application Priority Data
Dec. 20, 2020 (CN) 202011602292.8

(51) Int. Cl. C07K 16/28

(52) U.S. Cl. CPC C07K 16/2878; C07K 2317/24; C07K 2317/569; C07K 2317/92

(57) ABSTRACT

A BCMA-targeting single-domain antibody, which has high affinity for BCMA protein. A use of the single-domain antibody in preparing a medicament for preventing or treating a disease or condition.

### TECHNICAL FIELD

The present application relates to the field of biomedicine and, in particular, to a BCMA-targeting single-domain antibody.

### BACKGROUND

It has been found that there is a heavy-chain antibody (hcAb) composed of only heavy chains but with complete function in Bactrian camels, dromedaries, alpacas and llamas. The molecular weight of the variable domains of the hcAb (VHH for short) is only 1/10 of the molecular weight of a common antibody; it is now the smallest molecular fragment that can be obtained with complete antibody function and is referred to as a single-domain antibody (sdAb). Single-domain antibodies have advantages such as low immunogenicity, small molecular weight and great capacities to penetrate over other antibodies, and therefore they have wide application prospects in areas such as fundamental research, drug development and treatment of disease.

B-cell maturation antigen (BCMA) is a member of the tumor necrosis factor (TNF) receptor superfamily. It is expressed mainly in plasma cells and mature B lymphocytes and hardly in other normal tissues. A key feature of it is that it is highly expressed on all multiple myeloma cells.

BCMA has now become a very popular immunotherapeutic target for multiple myeloma and other hematologic malignancies, and therefore there is a need to develop novel anti-BCMA antibodies with high affinity and specificity for BCMA.

### SUMMARY

The present application provides an isolated antigen-binding fragment having one or more of the following properties: 1) being able to bind to BCMA protein with relatively high affinity and specificity. The present application also provides a method for preparing the isolated antigen-binding fragment and use thereof.

In one aspect, the present application provides an isolated antigen-binding fragment competing for binding to BCMA protein with a reference antibody, wherein the reference antibody comprises a heavy chain variable region; the heavy chain variable region of the reference antibody may comprise HCDR1, HCDR2 and HCDR3; the HCDR1 comprises amino acid sequences set forth in SEQ ID NO: 2 and SEQ ID NO: 9, the HCDR2 comprises amino acid sequences set forth in SEQ ID NO: 4 and SEQ ID NO: 11, and the HCDR3 comprises amino acid sequences set forth in SEQ ID NO: 6 and SEQ ID NO: 13.

In certain embodiments, the isolated antigen-binding fragment binds to BCMA protein with a K_{D} value that is exactly or about, or is less than about or less than, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM or 0.1 nM.

In certain embodiments, the isolated antigen-binding fragment includes a single-domain antibody.

In certain embodiments, the isolated antigen-binding fragment comprises a heavy chain variable region comprising HCDR1, HCDR2 and HCDR3, and the HCDR1 comprises amino acid sequences set forth in SEQ ID NO: 2 and SEQ ID NO: 9.

In certain embodiments, the HCDR2 comprises amino acid sequences set forth in SEQ ID NO: 4 and SEQ ID NO: 11.

In certain embodiments, the HCDR3 comprises amino acid sequences set forth in SEQ ID NO: 6 and SEQ ID NO: 13.

In certain embodiments, the isolated antigen-binding fragment comprises a heavy chain variable region comprising H-FR1, H-FR2, H-FR3 and H-FR4, and the H-FR1 comprises an amino acid sequence set forth in SEQ ID NO: 49 or SEQ ID NO: 50.

In certain embodiments, the heavy chain variable region in the isolated antigen-binding fragment comprises H-FR1, H-FR2, H-FR3 and H-FR4, and the H-FR1 comprises amino acid sequences set forth in SEQ ID NO: 1, SEQ ID NO: 8 and SEQ ID NO: 30.

In certain embodiments, the H-FR2 comprises an amino acid sequence set forth in SEQ ID NO: 51 or SEQ ID NO: 52.

In certain embodiments, the H-FR2 comprises amino acid sequences set forth in SEQ ID NO: 3, SEQ ID NO: 10 and SEQ ID NO: 33.

In certain embodiments, the H-FR3 comprises an amino acid sequence set forth in SEQ ID NO: 53 or SEQ ID NO: 54.

In certain embodiments, the H-FR3 comprises amino acid sequences set forth in SEQ ID NO: 5, SEQ ID NO: 12 and SEQ ID NO: 31.

In certain embodiments, the H-FR4 comprises an amino acid sequence set forth in SEQ ID NO: 55 or SEQ ID NO: 56.

In certain embodiments, the H-FR4 comprises amino acid sequences set forth in SEQ ID NO: 7, SEQ ID NO: 14 and SEQ ID NO: 32.

In certain embodiments, the heavy chain variable region comprises an amino acid sequence set forth in SEQ ID NO: 47 or SEQ ID NO: 48.

In certain embodiments, the heavy chain variable region comprises amino acid sequences set forth in SEQ ID NO: 15, SEQ ID NO: 16, SEQ ID NO: 20, SEQ ID NO: 21, SEQ ID NO: 22 and SEQ ID NO: 23.

In another aspect, the present application provides one or more isolated nucleic acid molecules comprising a polynucleotide encoding the isolated antigen-binding fragment described herein.

In certain embodiments, the isolated nucleic acid molecule comprises one or more polynucleotide sequences selected from the group consisting of SEQ ID NOs: 24-29.

In another aspect, the present application provides a vector comprising the isolated nucleic acid molecule described herein.

In another aspect, the present application provides a cell comprising the isolated nucleic acid molecule described herein or the vector described herein.

In another aspect, the present application provides a method for preparing the antigen-binding fragment comprising culturing the cell described herein under such conditions that the antigen-binding fragment is expressed.

In another aspect, the present application provides a pharmaceutical composition comprising the isolated antigen-binding fragment described herein, the isolated nucleic acid molecule described herein, the vector described herein and/or the cell described herein, and optionally a pharmaceutically acceptable adjuvant.

In another aspect, the present application provides use of the isolated antigen-binding fragment described herein, the nucleic acid molecule described herein, the vector described herein and/or the host cell described herein in preparing a medicament for preventing or treating a disease or condition.

In another aspect, the present application provides use of the isolated antigen-binding fragment described herein, the nucleic acid molecule described herein, the vector described herein and/or the host cell described herein in preparing a chimeric antigen receptor (CAR) and a chimeric antigen receptor T cell (CART).

Other aspects and advantages of the present application will be readily apparent to those skilled in the art from the following detailed description. Only exemplary embodiments of the present application have been shown and described in the following detailed description. As will be recognized by those skilled in the art, the content of the present application enables those skilled in the art to make changes to the specific embodiments disclosed without departing from the spirit and scope of the invention to which the present application pertains. Accordingly, descriptions in the drawings and specification are only illustrative rather than restrictive.

### BRIEF DESCRIPTION OF THE DRAWINGS

Specific features of the invention to which the present application pertains are set forth in appended claims. Features and advantages of the invention to which the present application pertains will be better understood by reference to the exemplary embodiments and drawings described in detail below. The drawings are briefly described as follows:

FIG. 1 is a graph showing the results of the two rounds of PCR amplification of VHH fragments as described herein.

FIG. 2 is a graph showing the results of the flow cytometry assays following streptavidin magnetic bead sorting as described herein.

FIG. 3 is a graph showing the flow cytometry analysis results of positive single clones of yeast (directly applied to PAD plates) induced to bind to Biotin-BCMA-Fc protein as described herein.

FIG. 4 is a graph showing the flow cytometry analysis results of BCMA-targeting single-domain antibodies binding to BCMA target protein as described herein.

FIG. 5 is a graph showing the results of the ligand (BCMA recombinant protein) coupling pre-enrichment experiment as described herein.

FIG. 6 is a graph showing the results of the ligand (BCMA recombinant protein) coupling experiment as described herein.

FIG. 7 is a graph showing the results of the single-domain antibodies 48-11/hu4811/hu4811FGLF binding to BCMA target protein as described herein.

FIG. 8 is a graph showing the results of the single-domain antibodies D1/huD1/huD1FGLF binding to BCMA target protein as described herein.

### Detailed Description of the Embodiments

The embodiments of the present invention are described below with reference to specific examples, and other advantages and effects of the present invention will be readily apparent to those skilled in the art from the disclosure of the present specification.

### Definitions of Terms

In the present application, the term “isolated antigen-binding fragment” generally refers to a portion of an intact antibody and the portion is capable of specifically recognizing and/or neutralizing a particular antigen. For example, the isolated antigen-binding fragment may comprise a portion of a heavy chain. For example, the isolated antigen-binding fragment may comprise a heavy chain variable region. The term “isolated antigen-binding fragment” may include single-domain antibodies, including but not limited to human single-domain antibodies.

In the present application, the term “single-domain antibody” generally refers to a class of antibodies that lack an antibody light chain and have only a heavy chain variable region. In certain cases, the single-domain antibody may be derived from Bactrian camels, dromedaries, alpacas, llamas, nurse sharks, smooth dogfishes or rays (see, e.g., Kang Xiaozhen et al., Chinese Journal of Biotechnology, 2018, 34 (12): 1974-1984). For example, the single-domain antibody may be derived from alpacas. The single-domain antibody may consist of a heavy chain variable region (VH). The term “heavy chain variable region” generally refers to the amino-terminal domain of the heavy chain of an antigen-binding fragment. The heavy chain variable region may be further divided into hypervariable regions termed complementarity-determining regions (CDRs), which are scattered over more conserved regions termed framework regions (FRs). Each heavy chain variable region may consist of three CDRs and four FRs arranged from the amino-terminus to the carboxyl-terminus in the following order: FR1, CDR1, FR2, CDR2, FR3, CDR3 and FR4. The heavy chain variable region comprises a binding domain that interacts with an antigen.

In the present application, the term “BCMA protein” generally refers to a biomarker that is expressed on B cells and is a member of the tumor necrosis factor (TNF) receptor superfamily. “BCMA protein” may also be referred to as TNFRSF17 or CD269. The amino acid sequence of human BCMA protein can be found under UniProt/Swiss-Prot accession No. Q02223. In the present application, the isolated antigen-binding fragment can bind to BCMA protein. In the present application, the terms “BCMA protein”, “BCMA antigen” and “BCMA-Fc recombinant protein” are used interchangeably and include any variant or isoform thereof that is naturally expressed by a cell.

In the present application, the term “framework region” (FR) generally refers to those variable domain residues other than CDR residues.

In the present application, the term “complementarity-determining region” (CDR) generally refers to a complementarity-determining region within a variable region of an antigen-binding fragment. In the present application, there are 3 CDRs present in the heavy chain variable region, and the CDRs are designated HCDR1, HCDR2 and HCDR3 for each variable region. The exact boundaries of those CDRs have been defined differently according to different systems. The system described by Kabat (Kabat et al., Sequences of Proteins of Immunological Interest, National Institutes of Health, Bethesda, Md. (1987) and (1991)) provides not only a clear residue numbering system applicable to any variable region of an antigen-binding fragment, but also precise residue boundaries defining 3 CDRs. Those CDRs may be referred to as Kabat CDRs. Chothia and colleagues (Chothia and Lesk, J. Mol. Biol., 196:901-917 (1987) and Chothia et al., Nature 342:877-883 (1989)) found that although there is large diversity at the amino acid sequence level, certain sub-portions within Kabat CDRs take almost identical peptide backbone conformations. Those sub-portions were designated L1, L2 and L3 or H1, H2 and H3, wherein “L” and “H” refer to the light and heavy chain regions, respectively. Those regions may be referred to as Chothia CDRs, which have boundaries that overlap with Kabat CDRs. Other boundaries defining CDRs that overlap with Kabat CDRs have been described by Padlan (FASEB J. 9:133-139 (1995)) and MacCallum (J Mol Biol 262 (5): 732-45 (1996)). In addition, other CDR boundary definitions may not strictly follow one of the above systems, but will nevertheless overlap with Kabat CDRs. Although they may be shortened or lengthened according to predictions or experimental findings that a particular residue or a particular group of residues or even the entire CDRs, do not significantly affect the antigen binding. In the present application, the IMGT numbering scheme is used.

In the present application, the term “homology” may generally be equivalent to sequence “identity”. A homologous sequence can include an amino acid sequence that can be at least 80%, 85%, 90%, 99.1%, 99.2%, 99.3%, 99.4%, 99.5%, 99.6%, 99.7%, 99.8%, or 99.9% identical to a subject sequence. Generally, the homolog will contain the same active sites as the amino acid sequence of the subject, etc. Homology may be considered in terms of similarity (i.e., amino acid residues having similar chemical properties/functions), or may be expressed in terms of sequence identity. In the present application, reference to a sequence having a percent identity of any one of the SEQ ID NOs of an amino acid sequence or a nucleotide sequence refers to a sequence having the percent identity over the entire length of the referenced SEQ ID NO.

To determine sequence identity, sequence alignments can be performed by various means known to those skilled in the art, e.g., using BLAST, BLAST-2, ALIGN, NEEDLE, or Megalign (DNASTAR) software, etc. Those skilled in the art can determine appropriate parameters for alignment, including any algorithms required to achieve optimal alignment over the full length of the sequences being compared.

In the present application, the term “KD” is used interchangeably with “KD” and generally refers to the dissociation equilibrium constant, in M (mol/L), of a particular antibody-antigen interaction. KD can be calculated from the concentration of substance AB and the concentration of substance A and substance B resulting from its dissociation: KD=c(A)×c(B)/c(AB). It can be seen from this equation that a larger KD indicates more dissociation and weaker affinity between substances A and B; conversely, a smaller KD indicates less dissociation and stronger affinity between substances A and B.

In the present application, the term “isolated nucleic acid molecule” generally refers to an isolated form of nucleotides, deoxyribonucleotides or ribonucleotides or analogs thereof of any length isolated from their natural environment, or artificially synthesized.

In the present application, the term “vector” generally refers to a nucleic acid molecule capable of self-replicating in a suitable host, which transfers an inserted nucleic acid molecule into a host cell and/or between host cells. The vector may include vectors primarily for the insertion of DNA or RNA into a cell, vectors primarily for the replication of DNA or RNA, and vectors primarily for the expression of transcription and/or translation of DNA or RNA. The vector also includes vectors having a variety of the above-described functions. The vector may be a polynucleotide capable of being transcribed and translated into a polypeptide when introduced into a suitable host cell. Generally, the vector can produce the desired expression product by culturing an appropriate host cell containing the vector.

In the present application, the term “host cell” generally refers to an individual cell, cell line or cell culture that may contain or has contained a vector comprising the isolated nucleic acid molecule described herein, or that is capable of expressing the isolated antigen-binding fragment described herein. The host cell may comprise progeny of a single host cell. Due to natural, accidental or deliberate mutations, progeny cells may not necessarily be identical in morphology or in genome to the original parent cell, but is capable of expressing the isolated antigen-binding fragment described herein. The host cell may be obtained by transfecting cells with the vector described herein in vitro. The host cell may be a prokaryotic cell (e.g., E. coli) or a eukaryotic cell (e.g., a yeast cell, a COS cell, a Chinese hamster ovary (CHO) cell, a HeLa cell, an HEK293 cell, a COS-1 cell, an NSO cell, or a myeloma cell). For example, the host cell may be an E. coli cell. For example, the host cell may be a yeast cell. For example, the host cell may be a mammalian cell. For example, the mammalian cell may be a CHO-K1 cell.

In the present application, the term “reference antibody” generally refers to an antibody that competes for binding to BCMA protein with the isolated antigen-binding fragment described herein. In certain cases, the isolated antigen-binding fragment has the same affinity, or at least substantially the same affinity, for BCMA as the reference antibody. In certain instances, the K_{D} value of the isolated antigen-binding fragment is approximately equal to or lower than the KD value of the reference antibody. In certain cases, the KD value of the isolated antigen-binding fragment is no more than about 2.5 times or no more than about 3 times, no more than 4 times and/or no more than 11 times greater than that of the reference antibody. In the present application, the term “comprise” or “comprising” generally means including, summarizing, containing or encompassing. In certain cases, the term also means “being” or “consisting of . . . ”.

In the present application, the term “about” generally means varying by 0.5%-10% above or below the stated value, for example, varying by 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% or 10% above or below the stated value.

### DETAILED DESCRIPTION OF THE INVENTION

### Antigen-Binding Fragment

The isolated antigen-binding fragment described herein can compete for binding to BCMA protein (e.g., human BCMA protein) with a reference antibody, wherein the reference antibody may comprise a heavy chain variable region; the heavy chain variable region of the reference antibody may comprise HCDR1, HCDR2 and HCDR3; in some embodiments, the HCDR1 comprises an amino acid sequence set forth in SEQ ID NO: 2, the HCDR2 comprises an amino acid sequence set forth in SEQ ID NO: 4, and the HCDR3 comprises an amino acid sequence set forth in SEQ ID NO: 6; in some other embodiments, the HCDR1 comprises an amino acid sequence set forth in SEQ ID NO: 9, the HCDR2 comprises an amino acid sequence set forth in SEQ ID NO: 11, and the HCDR3 comprises an amino acid sequence set forth in SEQ ID NO: 13.

For example, the heavy chain variable region of the reference antibody may comprise a sequence set forth in SEQ ID NO: 15. As another example, the heavy chain variable region of the reference antibody may comprise a sequence set forth in SEQ ID NO: 16.

The isolated antigen-binding fragment of the present application binds to BCMA protein with a KD value that is exactly or about, or is less than about or less than, 100 nM, 95 nM, 90 nM, 85 nM, 80 nM, 75 nM, 70 nM, 65 nM, 60 nM, 55 nM, 50 nM, 45 nM, 40 nM, 35 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM or 0.1 nM.

The isolated antigen-binding fragment described herein can compete for binding to BCMA protein with a BCMA ligand (e.g., B-cell activating factor BAFF or APRIL).

The BCMA protein described herein may include human BCMA protein. For example, the BCMA protein may comprise the amino acid sequence under UniProt/Swiss-Prot accession No. Q02223. For example, the BCMA protein may include a homolog of human BCMA protein. In the present application, the term “homolog” generally refers to an amino acid sequence or a nucleotide sequence having certain homology with the amino acid sequence of human BCMA protein and the nucleotide sequence of human BCMA protein. The term “homology” may be equivalent to sequence “identity”. A homologous sequence can include an amino acid sequence that can be at least 80%, 85%, 90%, 99.1%, 99.2%, 99.3%, 99.4%, 99.5%, 99.6%, 99.7%, 99.8%, or 99.9% identical to a subject sequence. Generally, the homolog will contain the same active sites as the amino acid sequence of the subject, etc. Homology may be considered in terms of similarity (i.e., amino acid residues having similar chemical properties/functions), or may be expressed in terms of sequence identity. In the present application, reference to a sequence having a percent identity of any one of the SEQ ID NOs of an amino acid sequence or a nucleotide sequence refers to a sequence having the percent identity over the entire length of the referenced SEQ ID NO. To determine sequence identity, sequence alignments can be performed by various means known to those skilled in the art, e.g., using BLAST, BLAST-2, ALIGN, NEEDLE, or Megalign (DNASTAR) software, etc. Those skilled in the art can determine appropriate parameters for alignment, including any algorithms required to achieve optimal alignment over the full length of the sequences being compared. In the present application, the terms “BCMA protein”, “BCMA antigen” and “BCMA-Fc recombinant protein” are used interchangeably.

In certain embodiments, the isolated antigen-binding fragment may be a single-domain antibody.

In certain embodiments, the isolated antigen-binding fragment comprises a heavy chain variable region comprising HCDR1, HCDR2 and HCDR3, wherein the HCDR1 comprises an amino acid sequence set forth in any one of SEQ ID NO: 2 and SEQ ID NO: 9; the HCDR2 comprises an amino acid sequence set forth in any one of SEQ ID NO: 4 and SEQ ID NO: 11; the HCDR3 comprises an amino acid sequence set forth in any one of SEQ ID NO: 6 and SEQ ID NO: 13.

For example, the HCDR1 comprises an amino acid sequence set forth in SEQ ID NO: 2, the HCDR2 comprises an amino acid sequence set forth in SEQ ID NO: 4, and the HCDR3 comprises an amino acid sequence set forth in SEQ ID NO: 6.

As another example, in some other embodiments, the HCDR1 comprises an amino acid sequence set forth in SEQ ID NO: 9, the HCDR2 comprises an amino acid sequence set forth in SEQ ID NO: 11, and the HCDR3 comprises an amino acid sequence set forth in SEQ ID NO: 13.

In certain embodiments, the isolated antigen-binding fragment comprises a heavy chain variable region comprising H-FR1, H-FR2, H-FR3 and H-FR4.

In some embodiments, the H-FR1 may comprise an amino acid sequence set forth in SEQ ID NO: 49 or SEQ ID NO: 50.

QVQLVESGGGLVQX₁GGSLRLSCAAS (SEQ ID NO: 49), where X₁ may be A or P.

QVQLVESGGGLVQX₁GGSLRLSCX₂AS (SEQ ID NO: 50), where X₁ may be A or P, and X₂ may be A or V.

For example, the H-FR1 may comprise an amino acid sequence set forth in any one of SEQ ID NO: 1, SEQ ID NO: 8 and SEQ ID NO: 30.

In some embodiments, the H-FR2 may comprise an amino acid sequence set forth in SEQ ID NO: 51 or SEQ ID NO: 52.

MGWFRQX₁PGKX₂X₃EFVAA (SEQ ID NO: 51), where X₁ may be A or T, X₂ may be E or G, and X₃ may be L or R.

MGWFRQAPGKX₁X₂X₃FVAA (SEQ ID NO: 52), where X₁ may be E or G, X₂ may be L or R, and X₃ may be E or R.

For example, the H-FR2 may comprise an amino acid sequence set forth in any one of SEQ ID NO: 3, SEQ ID NO: 10 and SEQ ID NO: 33.

In some embodiments, the H-FR3 may comprise an amino acid sequence set forth in SEQ ID NO: 53 or SEQ ID NO: 54.

YYX₁X₂SVKGRFTISRDNX₃KNTX₄YLQMNSLX₅X₆EDTAVYYC (SEQ ID NO: 53), where X₁ may be A or R, X₂ may be D or T, X₃ may be A or S, X₄ may be L or V, X₅ may be K or R, and X₆ may be A or P.

YYX₁X₂SVKGRFTISRDNX₃KNTX₄YLQMNSLX₅X₆EDTAVYYC (SEQ ID NO: 54), where X₁ may be A or G, X₂ may be D or E, X₃ may be D or S, X₄ may be L or V, X₅ may be K or R, and X₆ may be A or P.

For example, the H-FR3 may comprise an amino acid sequence set forth in any one of SEQ ID NO: 5, SEQ ID NO: 12 and SEQ ID NO: 31.

In some embodiments, the H-FR4 may comprise an amino acid sequence set forth in SEQ ID NO: 55 or SEQ ID NO: 56.

WGQGTX₁VTVSS (SEQ ID NO: 55), where X₁ may be L or Q.

WGQGTX₁VTVX₂S (SEQ ID NO: 56), where X₁ may be L or P, and X₂ may be P or S.

For example, the H-FR4 may comprise an amino acid sequence set forth in any one of SEQ ID NO: 7, SEQ ID NO: 14 and SEQ ID NO: 32.

As another example, in the isolated antigen-binding fragment, the H-FR1 may comprise an amino acid sequence set forth in SEQ ID NO: 1, the H-FR2 may comprise an amino acid sequence set forth in SEQ ID NO: 3, the H-FR3 may comprise an amino acid sequence set forth in SEQ ID NO: 5, and the H-FR4 may comprise an amino acid sequence set forth in SEQ ID NO: 7.

As another example, in the isolated antigen-binding fragment, the H-FR1 may comprise an amino acid sequence set forth in SEQ ID NO: 8, the H-FR2 may comprise an amino acid sequence set forth in SEQ ID NO: 10, the H-FR3 may comprise an amino acid sequence set forth in SEQ ID NO: 12, and the H-FR4 may comprise an amino acid sequence set forth in SEQ ID NO: 14.

As another example, in the isolated antigen-binding fragment, the amino acid sequence of the H-FR1 may include SEQ ID NO: 30, the amino acid sequence of the H-FR2 may include SEQ ID NO: 3, the amino acid sequence of the H-FR3 may include SEQ ID NO: 31, and the amino acid sequence of the H-FR4 may include SEQ ID NO: 32. For example, the isolated antigen-binding fragment may include antigen-binding fragment hu4811 or an antigen-binding fragment having the same H-FR1-4 as it.

As another example, in the isolated antigen-binding fragment, the amino acid sequence of the H-FR1 may include SEQ ID NO: 30, the amino acid sequence of the H-FR2 may include SEQ ID NO: 33, the amino acid sequence of the H-FR3 may include SEQ ID NO: 31, and the amino acid sequence of the H-FR4 may include SEQ ID NO: 32. For example, the isolated antigen-binding fragment may include antigen-binding fragment hu4811FGLF or an antigen-binding fragment having the same H-FR1-4 as it.

In certain embodiments, the heavy chain variable region comprises an amino acid sequence set forth in SEQ ID NO: 47 or SEQ ID NO: 48.

QVQLVESGGGLVQXIGGSLRLSCAASGHTPSNYAMGWFRQX₂PGKX₃X₄EFVAAITWSAVTRDDAIPYYX₅X₆SVKGRFTISRDNX-KNTX₇YLQMNSLX₉X₁₀EDTAVYYCAVDASLVMTPTPRYWGQGTX₁₁VTVSS (SEQ ID NO: 47), where X₁ may be A or P, X₂ may be A or T, X₃ may be E or G, X₄ may be L or R, X₅ may be A or R, X₆ may be D or T, X₇ may be A or S, X₈ may be L or V, X₉ may be K or R, X₁₀ may be A or P, and X₁₁ may be L or Q.

QVQLVESGGGLVQXIGGSLRLSCX₂ASGRTSTTSVMGWFRQAPGKX₃X₄X₅FVAAITRTDDRQNDGITYYX₆X-SVKGRFTISRDNX₈KNTX₉YLQMNSLX₁₀X₁₁EDTAVYYCAAHTSLVFTPDPRYWGQGTX₁₂VTVX₁₃S (SEQ ID NO: 48), where X₁ may be A or P, X₂ may be A or V, X₃ may be E or G, X₄ may be L or R, X₅ may be E or R, X₆ may be A or G, X₇ may be D or E, X₈ may be D or S, X₉ may be L or V, X₁₀ may be K or R, Xu may be A or P, X₁₂ may be L or P, and X₁₃ may be P or S.

In the present application, the heavy chain variable region may comprise an amino acid sequence set forth in any one of SEQ ID NO: 15, SEQ ID NO: 16, SEQ ID NO: 20, SEQ ID NO: 21, SEQ ID NO: 22 and SEQ ID NO: 23.

For example, the isolated antigen-binding fragment may comprise HCDR1-3 and H-FR1-4, and the amino acid sequence of HCDR1 may include SEQ ID NO: 2, the amino acid sequence of HCDR2 may include SEQ ID NO: 4, the amino acid sequence of HCDR3 may include SEQ ID NO: 6, the amino acid sequence of H-FR1 may include SEQ ID NO: 1, the amino acid sequence of H-FR2 may include SEQ ID NO: 3, the amino acid sequence of H-FR3 may include SEQ ID NO: 5, and the amino acid sequence of H-FR4 may include SEQ ID NO: 7. The antigen-binding fragment may comprise an amino acid sequence set forth in SEQ ID NO: 15 and is designated 48-11.

For example, the isolated antigen-binding fragment may comprise HCDR1-3 and H-FR1-4, and the amino acid sequence of HCDR1 may include SEQ ID NO: 2, the amino acid sequence of HCDR2 may include SEQ ID NO: 4, the amino acid sequence of HCDR3 may include SEQ ID NO: 6, the amino acid sequence of H-FR1 may include SEQ ID NO: 30, the amino acid sequence of H-FR2 may include SEQ ID NO: 3, the amino acid sequence of H-FR3 may include SEQ ID NO: 31, and the amino acid sequence of H-FR4 may include SEQ ID NO: 32. The antigen-binding fragment may comprise an amino acid sequence set forth in SEQ ID NO: 22 and is designated hu4811.

For example, the isolated antigen-binding fragment may comprise HCDR1-3 and H-FR1-4, and the amino acid sequence of HCDR1 may include SEQ ID NO: 2, the amino acid sequence of HCDR2 may include SEQ ID NO: 4, the amino acid sequence of HCDR3 may include SEQ ID NO: 6, the amino acid sequence of H-FR1 may include SEQ ID NO: 30, the amino acid sequence of H-FR2 may include SEQ ID NO: 33, the amino acid sequence of H-FR3 may include SEQ ID NO: 31, and the amino acid sequence of H-FR4 may include SEQ ID NO: 32. The antigen-binding fragment may comprise an amino acid sequence set forth in SEQ ID NO: 23 and is designated hu4811FGLF.

For example, the isolated antigen-binding fragment may comprise HCDR1-3 and H-FR1-4, and the amino acid sequence of HCDR1 may include SEQ ID NO: 9, the amino acid sequence of HCDR2 may include SEQ ID NO: 11, the amino acid sequence of HCDR3 may include SEQ ID NO: 13, the amino acid sequence of H-FR1 may include SEQ ID NO: 8, the amino acid sequence of H-FR2 may include SEQ ID NO: 10, the amino acid sequence of H-FR3 may include SEQ ID NO: 12, and the amino acid sequence of H-FR4 may include SEQ ID NO: 14. The antigen-binding fragment may comprise an amino acid sequence set forth in SEQ ID NO: 16 and is designated D1.

For example, the isolated antigen-binding fragment may comprise HCDR1-3 and H-FR1-4, and the amino acid sequence of HCDR1 may include SEQ ID NO: 9, the amino acid sequence of HCDR2 may include SEQ ID NO: 11, the amino acid sequence of HCDR3 may include SEQ ID NO: 13, the amino acid sequence of H-FR1 may include SEQ ID NO: 30, the amino acid sequence of H-FR2 may include SEQ ID NO: 10, the amino acid sequence of H-FR3 may include SEQ ID NO: 31, and the amino acid sequence of H-FR4 may include SEQ ID NO: 32. The antigen-binding fragment may comprise an amino acid sequence set forth in SEQ ID NO: 20 and is designated huD1.

For example, the isolated antigen-binding fragment may comprise HCDR1-3 and H-FR1-4, and the amino acid sequence of HCDR1 may include SEQ ID NO: 9, the amino acid sequence of HCDR2 may include SEQ ID NO: 11, the amino acid sequence of HCDR3 may include SEQ ID NO: 13, the amino acid sequence of H-FR1 may include SEQ ID NO: 30, the amino acid sequence of H-FR2 may include SEQ ID NO: 33, the amino acid sequence of H-FR3 may include SEQ ID NO: 31, and the amino acid sequence of H-FR4 may include SEQ ID NO: 32. The antigen-binding fragment may comprise an amino acid sequence set forth in SEQ ID NO: 21 and is designated huD1FGLF.

The protein, polypeptide and/or amino acid sequence involved in the present application is also to be understood as including at least the following ranges: variants or homologs having the same or similar function as the protein or polypeptide.

In the present application, the variants may be proteins or polypeptides derived from the protein and/or the polypeptide (e.g., an antigen-binding fragment that specifically binds to BCMA protein) by substituting, deleting or adding one or more amino acids. For example, the functional variants may include proteins or polypeptides having amino acid changes by substituting, deleting and/or inserting at least 1, e.g., 1-30, 1-20 or 1-10, or 1, 2, 3, 4 or 5, amino acids. The functional variants may substantially retain the biological properties of the protein or the polypeptide before the changes (e.g., substitutions, deletions or additions). For example, the functional variants may retain at least 60%, 70%, 80%, 90% or 100% of the biological activity (e.g., antigen-binding capacity) of the protein or the polypeptide before the changes. For example, the substitutions may be conservative.

In the present application, the homologs may be proteins or polypeptides having at least about 85% (e.g., having at least about 85%, about 90%, about 91%, about 92%, about 93%, about 94%, about 95%, about 96%, about 97%, about 98%, about 99% or higher) sequence homology with the protein and/or the polypeptide (e.g., an antigen-binding fragment that specifically binds to BCMA protein).

In the present application, the homology generally refers to similarity, likeness or association between two or more sequences. “Percent sequence homology” can be calculated by the following steps: comparing two sequences to be aligned in a comparison window; determining the number of positions at which nucleic acid bases (e.g., A, T, C, G and I) or amino acid residues (e.g., Ala, Pro, Ser, Thr, Gly, Val, Leu, Ile, Phe, Tyr, Trp, Lys, Arg, His, Asp, Glu, Asn, Gln, Cys and Met) are identical in the two sequences to give the number of matched positions; dividing the number of matched positions by the total number of positions in the comparison window (i.e., the window size); and multiplying the result by 100 to give a percent sequence homology. Alignment for determining the percent sequence homology can be achieved in a variety of ways known in the art, for example, using publicly available computer software such as BLAST, BLAST-2, ALIGN or Megalign (DNASTAR) software. Those skilled in the art can determine suitable parameters for alignment of the sequences, including any algorithms required to achieve optimal alignment in a full-length sequence range or target sequence region being compared. The homology can also be determined by the following methods: FASTA and BLAST. For the description of the FASTA algorithm, see W. R. Pearson and D. J. Lipman, “Improved Tools for Biological Sequence Comparison”, Proc. Natl. Acad. Sci., 85:2444-2448, 1988; and D. J. Lipman and W. R. Pearson, “Rapid and Sensitive Protein Similarity Searches”, Science, 227:1435-1441, 1989. For the description of the BLAST algorithm, see S. Altschul, W. Gish, W. Miller, E. W. Myers and D. Lipman, “A Basic Local Alignment Search Tool”, Journal of Molecular Biology, 215:403-410, 1990.

Nucleic acids, vectors, host cells and preparation methods

In another aspect, the present application also provides one or more isolated nucleic acid molecules that can encode the isolated antigen-binding fragment described herein. For example, the one or more nucleic acid molecules can each encode the antigen-binding fragment in its entirety or a portion of it (e.g., one or more of HCDR1-3 and the heavy chain variable region).

The nucleic acid molecules described herein may be isolated. For example, they may be produced or synthesized by the following methods: (i) in vitro amplification, such as amplification by polymerase chain reaction (PCR); (ii) cloning and recombination; (iii) purification, such as separation by enzymatic digestion and gel electrophoresis fractionation; or (iv) synthesis, such as chemical synthesis. For example, the isolated nucleic acids may be nucleic acid molecules prepared by recombinant DNA techniques.

In the present application, the nucleic acids encoding the isolated antigen-binding fragment can be prepared by a variety of methods known in the art, including but not limited to using reverse transcription PCR and PCR to obtain the nucleic acid molecules for the isolated antigen-binding fragment described herein.

In another aspect, the present application provides one or more vectors comprising the one or more nucleic acid molecules described herein. Each of the vectors may comprise one or more of the nucleic acid molecules. In addition, the vectors may further comprise other genes, e.g., marker genes that allow the selection of the vectors in an appropriate host cell and under appropriate conditions. In addition, the vectors may further comprise expression control elements that allow the proper expression of coding regions in an appropriate host. Such control elements are well known to those skilled in the art and may include, for example, promoters, ribosome binding sites, enhancers and other control elements for regulating gene transcription or mRNA translation. In certain embodiments, the expression control sequences are adjustable elements. The specific structures of the expression control sequences may vary depending on the functionality of the species or cell type but generally comprise 5′ non-transcribed sequences and 5′ and 3′ non-translated sequences which are involved in the initiation of transcription and translation, respectively, e.g., TATA boxes, capping sequences and CAAT sequences. For example, the 5′ non-transcribed expression control sequences may comprise a promoter region, which may include a promoter sequence for transcriptional control of the functionally linked nucleic acid. The expression control sequences may also include enhancer sequences or upstream activator sequences. In the present application, suitable promoters may include, for example, promoters for SP6, T3 and T7 polymerase, human U6RNA promoter, CMV promoter, and artificial hybrid promoters thereof (e.g., CMV), wherein a portion of the promoter may be fused to a portion of promoters of genes of other cellular proteins (such as human GAPDH and glyceraldehyde-3-phosphate dehydrogenase), which may include or not include additional introns. The one or more nucleic acid molecules described herein may be operably linked to the expression control elements.

The vectors may include, for example, plasmids, cosmids, viruses, phages or other vectors commonly used in, for example, genetic engineering. For example, the vectors are expression vectors.

In another aspect, the present application provides host cells that may comprise the one or more nucleic acid molecules described herein and/or the one or more vectors described herein. For example, each type of or each of the host cells may comprise one type of or one of the nucleic acid molecules or vectors described herein. For example, each type of or each of the host cells may comprise several (e.g., two or more) of or several types (e.g., two or more types) of the nucleic acid molecules or vectors described herein. For example, the vectors described herein can be introduced into the host cells, e.g., prokaryotic cells (e.g., bacterial cells), CHO cells, NS/0 cells, HEK293T cells or HEK293A cells, or other eukaryotic cells, such as plant-derived cells or fungal or yeast cells. The vectors described herein can be introduced into the host cells described herein using methods known in the art, such as electroporation, lipofectine transfection and lipofectamin transfection. For example, the host cells may be yeast cells. For example, the host cells may be E. coli cells. For example, the host cells may be mammalian cells. For example, the mammalian cells may be CHO-K1.

In another aspect, the present application provides a method for preparing the isolated antigen-binding fragment described herein. The method may comprise culturing the host cells described herein under such conditions that the isolated antigen-binding fragment is expressed.

For example, an appropriate culture medium, an appropriate temperature, an appropriate incubation time, and the like, may be adopted. These methods are known to those of ordinary skill in the art.

In certain cases, the method may further comprise the step of harvesting (e.g., isolating and/or purifying) the isolated antigen-binding fragment described herein. For example, affinity chromatography can be performed with protein G-agarose or protein A-agarose; the isolated antigen-binding fragment described herein can also be purified and isolated by gel electrophoresis and/or high performance liquid chromatography, etc. For example, the fusion protein polypeptide bound to the affinity column can also be eluted by using a high-salt buffer, changing the pH, etc.

### Pharmaceutical Composition and Use

In another aspect, the present application provides a pharmaceutical composition comprising the isolated antigen-binding fragment described herein, and optionally a pharmaceutically acceptable carrier.

In the present application, the pharmaceutically acceptable carrier may include a buffer, an antioxidant, a preservative, a low molecular weight polypeptide, a protein, a hydrophilic polymer, an amino acid, a sugar, a chelating agent, a counter ion, a metal complex, and/or a nonionic surfactant, and the like.

In the present application, the pharmaceutical composition may be formulated for oral administration, intravenous administration, intramuscular administration, in situ administration at the tumor site, inhalation, rectal administration, vaginal administration, transdermal administration or administration via a subcutaneous depot.

For example, the pharmaceutical composition may be used to inhibit or delay the development or progression of a disease or condition, and/or may alleviate and/or stabilize the state of the disease or condition.

The pharmaceutical composition described herein may comprise a prophylactically and/or therapeutically effective amount of the isolated antigen-binding fragment.

The prophylactically and/or therapeutically effective amount is the dose required to prevent and/or treat (at least partially treat) a disease or condition and/or any complication thereof in a subject suffering from or at risk of developing the disease or condition.

In another aspect, the present application provides use of the isolated antigen-binding fragment in preparing a medicament for preventing or treating a disease or condition.

In another aspect, the isolated antigen-binding fragment provided herein can be used for preventing or treating a disease or condition.

In another aspect, the present application provides a method for preventing or treating a disease or condition comprising administering (e.g., to a subject in need thereof) the isolated antigen-binding fragment described herein, the nucleic acid molecules described herein, the vectors described herein, the host cells described herein and/or the pharmaceutical composition described herein.

The isolated antigen-binding fragment described herein can ameliorate or treat a disease or condition associated with BCMA overexpression. For example, the disease or condition may be selected from the group consisting of multiple myeloma, Hodgkin lymphoma and autoimmune diseases.

In another aspect, the present application provides a method for inhibiting BCMA protein from binding to ligands for BCMA comprising administering the isolated antigen-binding fragment described herein, the nucleic acid molecules described herein, the vectors described herein, the host cells described herein and/or the pharmaceutical composition described herein. For example, the method may be an in vitro or ex vivo method.

Without being bound by any theory, the following examples are intended only to illustrate the antigen-binding fragment, preparation method, use, etc., of the present application, and are not intended to limit the scope of the present application.

### EXAMPLES

### Example 1. Immunization of Alpaca

1.1. The prepared BCMA-Fc recombinant protein was used to perform immunization on an alpaca six times. The serum of the immunized alpaca was isolated and tested by ELISA to check the immunization result. According to the titer results, rush immunization was performed. Ten days after the rush immunization, peripheral blood was collected and PBMCs were isolated from it to construct a cell bank.

### Example 2. Immunization Titer Tests

2.1. Into a centrifuge tube, 5 mL of peripheral blood was collected, and the tube was placed in a 37° C. incubator and left to stand for 1 h. Then the peripheral blood was transferred to a 4° C. environment and left to stand overnight, and the serum of the immunized alpaca was isolated.
2.2. The isolated serum of the immunized alpaca was transferred to a new sterile centrifuge tube, centrifuged at 5000 rpm for 20 min, diluted by limiting dilution in ratios 1:1 k, 1:2 k, 1:4 k, 1:8 k, 1:16 k, 1:32 k and 1:64 k and used in a 96-well plate pre-coated with BCMA-Fc recombinant protein to perform immunization titer tests via ELISA. The results are shown in Table 1. According to the ELISA results, the BCMA-Fc recombinant protein immunization titer was high.

TABLE 1
The ELISA results of the immunization titer

| Serum dilution ratio | OD value |
|---|---|
| 1:1k | 2.866 |
| 1:2k | 2.744 |
| 1:4k | 2.498 |
| 1:8k | 2.14 |
| 1:16k | 1.748 |
| 1:32k | 1.2 |
| 1:64k | 0.768 |
| PBS | 0.072 |

### Example 3. Biotin-Conjugated Antigen Protein

3.1. 1 mg of BCMA-Fc recombinant protein was prepared; the buffer system was PBS, and the concentration was 1 mg/mL.
3.2. NHS-biotin was measured out and dissolved in DMSO to prepare 10 mM NHS-biotin.
3.3. To the above BCMA-Fc recombinant protein was added the freshly prepared 10 mM NHS-biotin solution. The sample tube was placed in a lightproof zipper bag. Conjugation was allowed at room temperature at 180 RPM for 30 min.
3.4. PBS exchange was performed to remove unlabeled Biotin. The labeled BCMA-Fc recombinant protein was stored at −80° C.
3.5. The Biotin conjugation result was checked using an ELISA scheme.

### Example 4. PBMC Isolation and Production of VHH Antibody Fragments

4.1. 150 mL of peripheral blood was collected and PBMCs were isolated from it using a lymphocyte isolation reagent.
4.2. The RNA was extracted and reverse-transcribed using PrimeScript™ II 1st Strand cDNA Synthesis Kit to prepare cDNA.
1) The following reaction mixture Mix1 (shown in Table 2) was prepared in a 200 μL PCR tube:

TABLE 2
Reaction mixture Mix1

| Reagent | Amount |
|---|---|
| Oligo dT Primer (50 μM) | 8 μL |
| dNTP Mixture (10 mM each) | 8 μL |
| Total RNA sample | 20 μg |
| RNase-free water | up to 80 μL |

2) The reaction mixture was incubated at 65° C. for 5 min and then rapidly cooled.
3) The following reaction mixture (shown in Table 3) was prepared in the above PCR tube:

TABLE 3
The components of the reaction mixture

| Reagent | Amount |
|---|---|
| The denatured reaction mixture described above | 80 μL |
| 5 × PrimeScript II Buffer | 32 μL |
| RNase Inhibitor (40 M/μL) | 4 μL |
| PrimeScript II RTase (200 M/μL) | 8 μL |
| RNase-free water | 36 μL |

4) The reaction mixture was well mixed by pipetting and aliquoted at 80 μL/tube. The aliquots were placed in a PCR instrument and left to stand at 42° C. for 1 h and thermally inactivated at 70° C. for 15 min. Finally, the cDNA samples were placed on ice or stored at −20° C. for a long time.
4.3. Amplification of VHH fragments

Preparation of the reaction system for the first round of PCRs (50 μL/tube, shown in Table 4):

TABLE 4
The reaction system for the first round of PCRs

| Component | Amount |
|---|---|
| Upstream primer (5 μM) | 2 μL |
| Downstream primer (10 μM) | 1 μL |
| NuHi Power mix (2×) | 25 μL |
| cDNA template | 2 μL |
| Sterile water | 20 μL |

2) After the PCR reaction system was prepared, the PCR instrument was programmed as follows (shown in Table 5):

TABLE 5
PCR program

| Program | Temperature | Time | Number of cycles |
|---|---|---|---|
| Pre-denaturation | 95° C. | 10 min | |
| Denaturation | 95° C. | 15 s | 30× |
| Annealing | 55° C. | 30 s | |
| Extension | 68° C. | 1 min | |
| Final extension | 68° C. | 10 min | |

3) Agarose electrophoresis of the PCR products

The PCR products were analyzed by electrophoresis using 1% agarose, and fragments having a molecular weight of about 750 bp were separated. The PCR products were recovered using a gel recovery kit and the concentration was determined using NanoDrop.
4) Preparation of the reaction system for the second round of PCRs (50 μL/tube, shown in Table 6):

TABLE 6
The reaction system for the second round of PCRs

| Component | Amount |
|---|---|
| 2^{nd} F primer | 2 μL |
| 2^{nd} R primer | 2 μL |
| NuHi Power mix (2×) | 25 μL |
| Products recovered from the first round of PCRs | 200 ng |
| Sterile water | Making up to |
| | 50 μL |

5) After the PCR reaction system was prepared, the PCR instrument was programmed as follows (shown in Table 7):

TABLE 7
PCR program

| Program | Temperature | Time | Number of cycles |
|---|---|---|---|
| Pre-denaturation | 95° C. | 10 min | |
| Denaturation | 95° C. | 15 s | 25× |
| Annealing | 55° C. | 30 s | |
| Extension | 68° C. | 1 min | |
| Final extension | 68° C. | 10 min | |

6) Agarose electrophoresis analysis of the products of the second round of PCRs

The PCR products were analyzed by electrophoresis using 1% agarose, and VHH fragments having a molecular weight of about 400 bp were separated. The VHH PCR products were recovered using a gel recovery kit and the concentration was determined using NanoDrop.

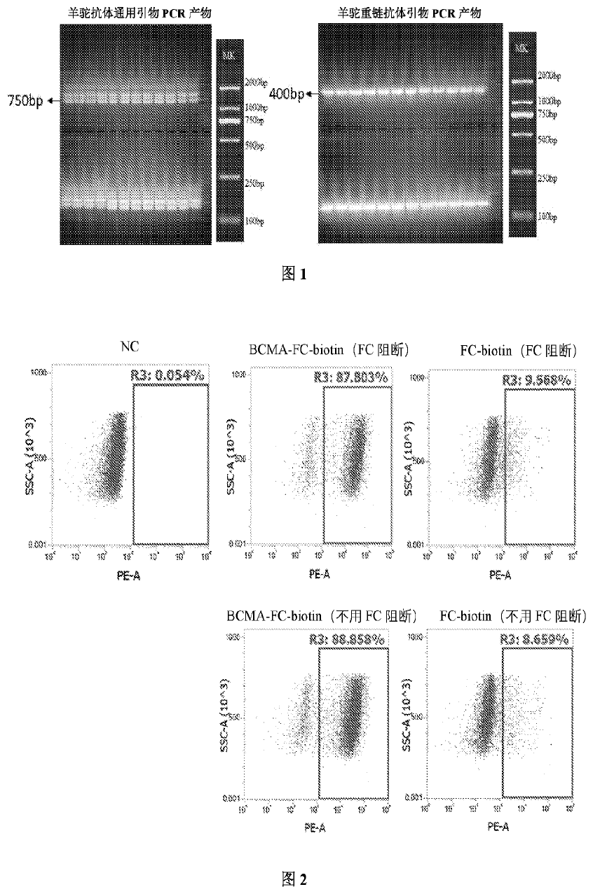

As shown in FIG. 1, PCR bands (without CHI region) of about 1000 bp and 750 bp were obtained from the first round of PCRs, and the 750 bp fragments were recovered by gel recovery as the template for the second round of PCRs. A band of about 400 bp was obtained as VHH fragments from the second round of PCRs.

### Example 5. Construction of Yeast Display Library

5.1. Construction of yeast display vectors
1) pBlue vectors and the VHH fragments obtained above were digested with the enzyme Sfil at 50° C. overnight.
2) The fragments of the pBlue vectors were isolated using 1% agarose gel, and 9000 bp vector fragments were recovered by gel recovery; meanwhile, the digested PCR products were purified using a DNA fragment recovery kit, and the concentration was determined using NanoDrop.
3) The digested pBlue vectors and VHH fragments were ligated using T4 ligase at 16° C. overnight to obtain yeast display vectors.
5.2 Production of E. coli library by electroporation of yeast display vectors
1) Electroporation cuvettes, the yeast display vectors and electroporation-competent cells were prepared and pre-cooled on ice.
2) The pre-cooled yeast display vectors were added to the electroporation-competent cells, and the mixture was left to stand on ice for 1 min. To each of the electroporation cuvettes was added 70 μL of the mixture of yeast display vectors/electroporation-competent cells, and the electroporation cuvettes were placed on ice.
3) Electroporation was carried out at 2500 V for 5 ms.
4) Following the electric shock, an SOC medium equilibrated to room temperature was immediately added to resuspend the bacterial cells. The cells were cultured on a shaker at 37° C. for 1 h.
5) 20 μL of the bacterial liquid was measured out for capacity QC and diversity QC, and the remaining bacterial liquid was inoculated to an LB medium with Amp resistance. The cells were selectively cultured at 37° C. at 180 rpm overnight.
6) The bacterial liquid cultured overnight was centrifuged, and the bacterial cell pellet was collected. Plasmids were extracted in large amounts using a plasmid maxi kit. The concentration of plasmids in the E. coli library was determined using Nanodrop, and the total amount of plasmids in the E. coli library was calculated.

### Example 6. Production of Yeast Display Library by Electroporation

6.1. The Pmel linearization of the E. coli library plasmids was performed. The enzyme digestion system is shown in Table 8:

TABLE 8
Enzyme digestion system

| Reagent | Amount |
|---|---|
| E. coli library plasmid | 12 μg |
| 10 × buffer | 5 μL |
| PmeI | 1 μL |
| Sterile water | up to |
| | 50 μL |

6.2 The E. coli library plasmids were digested at 37° C. for 3 h; 5 μL of the digestion product was measured out and assayed by 1% agarose electrophoresis, and the remaining digestion product was precipitated and concentrated for later use. A total of 1 mg of E. coli library plasmids was digested.
6.3 Preparation of competent cells of yeast
1) Yeast cells were picked from yeast glycerol stock and inoculated onto a YPD agar plate by streaking. The plate was placed in a 24-30° C. incubator and the cells were continuously cultured for 3-5 days until single clones were produced.
2) Single clones of yeast were picked from the plate, added to a liquid YPD medium, placed on a 24-30° C. shaker at 250 RPM and cultured for 1-2 days.
3) To a 1 L sterile Erlenmeyer flask was added 100 mL of YPD medium, followed by the above-prepared shaken bacterial product. The flask was placed in a 24-30° C. shaker at 250 RPM and the product was cultured for 1-2 days.
4) The culture was sampled for OD₆₀₀ until it was 1.3-1.5 (log phase).
5) The bacterial liquid was transferred in its entirety to a centrifuge tube and centrifuged at 1500×g at 4° C. for 5 min. After the supernatant was removed, the bacterial cell pellet was resuspended in 250 mL of sterile water that had been pre-cooled on ice.
6) After another round of centrifugation at 1500×g at 4° C. for 5 min, the supernatant was removed and the bacterial cell pellet was resuspended in 50 mL of sterile water that had been pre-cooled on ice.
7) After another round of centrifugation at 1500×g at 4° C. for 5 min, the supernatant was removed and the bacterial cell pellet was resuspended in 10 mL of 1 M sorbitol that had been pre-cooled on ice.
8) After another round of centrifugation at 1500×g at 4° C. for 5 min, the supernatant was removed and the bacterial cell pellet was resuspended in 500 μL of 1 M sorbitol that had been pre-cooled on ice.
9) Finally, 500 μL of competent cells of yeast was obtained and aliquoted at 80 μL/tube for later use. Competent cells are generally prepared freshly when needed in order to ensure higher electroporation efficiency.
6.4. Electroporation of competent cells of yeast
1) 80 μL of competent cells of yeast was measured out and 1 μg of linearized E. coli library plasmids was added. They were well mixed and then added to an electroporation cuvette that had been pre-cooled on ice, and the cuvette was placed on ice and cooled in the ice bath for 5 min.
2) Electroporation was performed under the following conditions: voltage: 1500 V; time: 5 ms, 2 electric shocks.
3) Following the shocks, 1 mL of YPDS medium that had been pre-cooled on ice was immediately added. After the contents were well mixed by gentle pipetting, the liquid in the cuvette was transferred in its entirety to a new EP tube and cultured standing in a 30° C. incubator for 2 h.
4) After incubation, 50 μL of the electroporation product was measured out and uniformly applied onto a PAD selective plate. The plate was placed in a 28° C. incubator and incubated for 3 days until single clones were produced.
5) The remaining transformation product was transferred to a liquid PAD medium and cultured with shaking at 28° C. for 3 days.
6) When it was observed that white bacterial cells had been predominant, the bacterial cells were collected by centrifugation at 1500×g for 5 min and a part of them was taken for induced expression of a yeast display library. The remaining cells were preserved in 50% sterile glycerol at −80° C.

### Example 7. Induced Expression and Sorting of Yeast Display Libraries

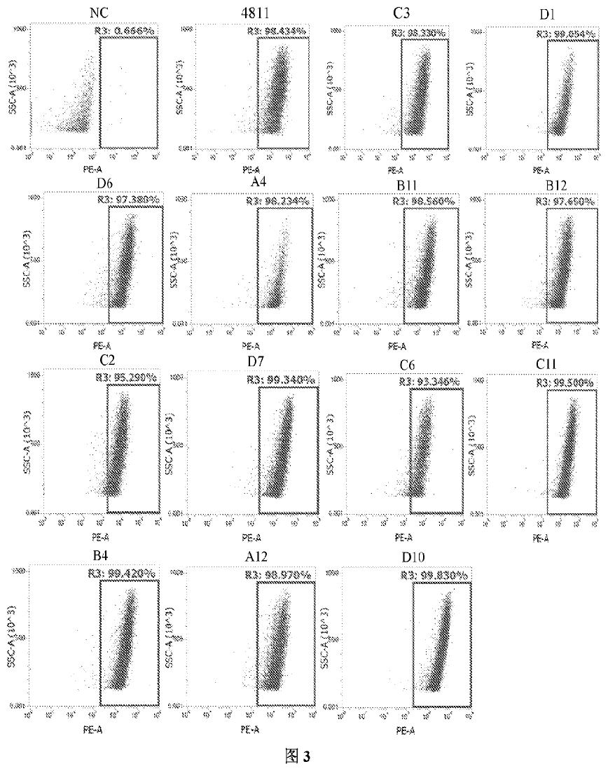

7.1. Induced expression of yeast display libraries
1) The yeast selectively grown in the PAD liquid medium was washed once with sterile PBS and then resuspended in a BMMY medium, and ampicillin and kanamycin antibiotics were added. Induced expression was performed at 28° C. at 220 rpm for 24-48 h.
2) Before induction, 1 mL of bacterial liquid was measured out and added to a YPD medium, and ampicillin and kanamycin antibiotics were added. The cells were cultured with shaking at 28° C. at 220 rpm, as a pre-induction control sample.
7.2 Magnetic sorting of yeast display libraries
1) Pre-adsorption by streptavidin magnetic beads: 10 OD induction yeast was resuspended in 500 μL of pre-cooled 0.5% PBSA. 50 μL of streptavidin magnetic beads was measured out and well mixed by pipetting, and 1 mL of PBSA was then added. The mixture was placed on a magnetic rack and left to stand for 3-5 min, and the supernatant was carefully removed. 1 mL of PBSA was added to wash the magnetic beads once again. The supernatant was pipetted off, and the yeast of step 1) was added. The mixture was well mixed, incubated at 4° C. for 1 h, placed on a magnetic rack and left to stand for 3-5 min. The supernatant was carefully pipetted off, and the yeast was transferred to a new EP tube. Adsorption was performed once again, and the supernatant was carefully pipetted off. The yeast was used for the next step.
2) Biotin-Fc negative sorting: The pre-adsorbed yeast was centrifuged at 800 g for 5 min and resuspended in Biotin-Fc. The suspension was incubated at 4° C. for 1 h. To the incubated yeast was added 1 mL of PBSA. The mixture was centrifuged at 800 g for 5 min, and the supernatant was discarded. Washing was repeated twice, and the yeast was finally resuspended in 500 μL of PBS. 50 μL of streptavidin magnetic beads was measured out and well mixed by pipetting, and 1 mL of PBSA was then added. The mixture was placed on a magnetic rack and left to stand for 3-5 min, and the supernatant was carefully removed. 1 mL of PBSA was added to wash the magnetic beads once again. The supernatant was pipetted off, and the Biotin-Fc-incubated yeast was added. The mixture was well mixed, incubated at 4° C. for 1 h, placed on a magnetic rack and left to stand for 3-5 min. The supernatant was carefully pipetted off, and the yeast was transferred to a new EP tube. Adsorption was performed once again, and the supernatant was carefully pipetted off. The residue was centrifuged at 800 g for 5 min, and the yeast was collected for use in the next step.
3) Biotin-BCMA-Fc magnetic sorting: The yeast obtained by Biotin-Fc negative sorting was centrifuged at 800 g for 5 min and resuspended in Biotin-BCMA-Fc. The suspension was incubated at 4° C. for 1 h. To the incubated yeast was added 1 mL of PBSA. The mixture was centrifuged at 800 g for 5 min, and the supernatant was discarded. Washing was repeated twice, and the yeast was finally resuspended in 500 μL of PBS. 100 μL of streptavidin magnetic beads was measured out and well mixed by pipetting, and 1 mL of PBSA was then added. The mixture was placed on a magnetic rack and left to stand for 3-5 min, and the supernatant was carefully removed. 1 mL of PBSA was added to wash the magnetic beads once again. The supernatant was pipetted off, and the Biotin-BCMA-Fc-incubated yeast was added. The mixture was well mixed and incubated at 4° C. for 1 h. After the incubation, the mixture was placed on a magnetic rack and left to stand for 3-5 min, and the supernatant was carefully pipetted off. 1 mL of PBSA was added to repeatedly wash the magnetic beads twice. The yeast was resuspended in a YPD medium, and culture and induced expression were performed. The flow cytometry analysis results are shown in FIG. 2: whether Fc blocking was performed or not, the yeast display library of the secondary magnetic sorting reacted with Fc and substantially did not bind to it; the binding of the yeast display library of the secondary magnetic sorting to BCMA-Fc before Fc blocking was consistent with that after Fc blocking, which indicates that positive yeast clones that specifically bind to BCMA can be obtained by positive yeast clone screening using the scheme combining streptavidin magnetic bead sorting and Fc removal. The Fc-bound clones were removed and single clones were selected.
7.3. Flow cytometry assays for single clones of yeast

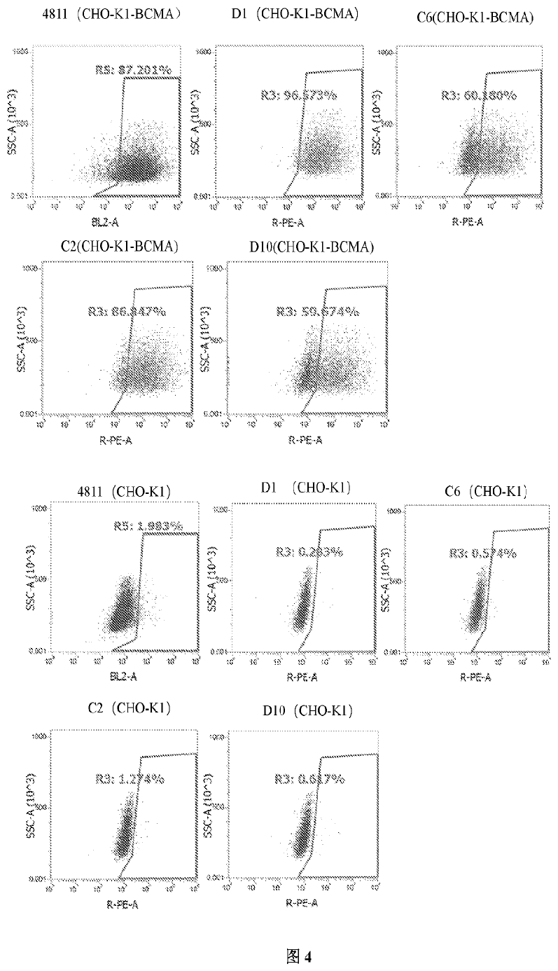

After two rounds of magnetic sorting were repeated, a part of the yeast was cultured and subjected to induced expression and flow cytometry assays. Another part was directly applied onto a PAD plate, and single clones were then picked and cultured, and were incubated simultaneously with Biotin-Fc and Biotin-BCMA-Fc 24 h after induced expression, with PE-Streptavidin as a secondary antibody; after incubation, flow cytometry assays were performed, and the results are shown in FIG. 3, where 14 clones are shown to bind to Biotin-BCMA-Fc.

### Example 8. Construction of Eukaryotic Expression Vectors for VHH

8.1. According to the flow cytometry assay results for the single clones of yeast, positive clones with varying abilities to bind to the target antigen were selected, and the genomic DNA was extracted and amplified by PCR using the universal primer of the pBlue vector. The PCR products were sequenced, and VHH antibody sequences were obtained. The VHH antibody sequences obtained by analysis were each genetically synthesized, ligated to human IgG1Fc in series and subcloned into the expression vector Lenti-hIgG1-Fc2. After the recombinant antibody expression vector Lenti-hIgG1-Fc2 passed sequencing validation, endotoxin-free plasmids were prepared for later use using a Qiagen plasmid maxi kit.

### Example 9. Expression of Recombinant Antibodies

9.1. The LVTransm transfection reagent and the recombinant antibody expression vector Lenti-hIgG1-Fc2 were taken out of the refrigerator, thawed at room temperature and pipetted up and down until they were each well mixed. PBS buffer was taken out and warmed to room temperature. To a well of a 24-well plate was added 500 μL of PBS followed by 4 μg of the recombinant antibody expression vector Lenti-hIgG1-Fc2. The mixture was pipetted up and down until it was well mixed, and 12 μL of LVTransm was then added. The mixture was immediately pipetted up and down until it was well mixed, and was left to stand at room temperature for 10 min. This mixture is referred to as a DNA/LVTransm complex.
9.2. To 1.5 mL of 293F cells was added 532 μL of the DNA/LVTransm complex, and they were well mixed by gentle shaking. The cells were placed in a 37° C., 5% CO₂ incubator and cultured at 130 RPM for 6-8 h, and 1.5 mL of fresh FreeStyle™ 293 medium was then added. The cells were placed back into the incubator and cultured again.
9.3. After 3 days of continuous culture, the culture was centrifuged, and the medium supernatant was collected and filtered through a 0.45 μm filter membrane. The filtrate was transferred to a sterile centrifuge tube; the recombinant antibodies in the filtrate were single-domain antibodies. Flow cytometry and ELISA assays were performed subsequently.

### Example 10. Flow Cytometry Assays for Binding of Single-Domain Antibodies to Target Protein

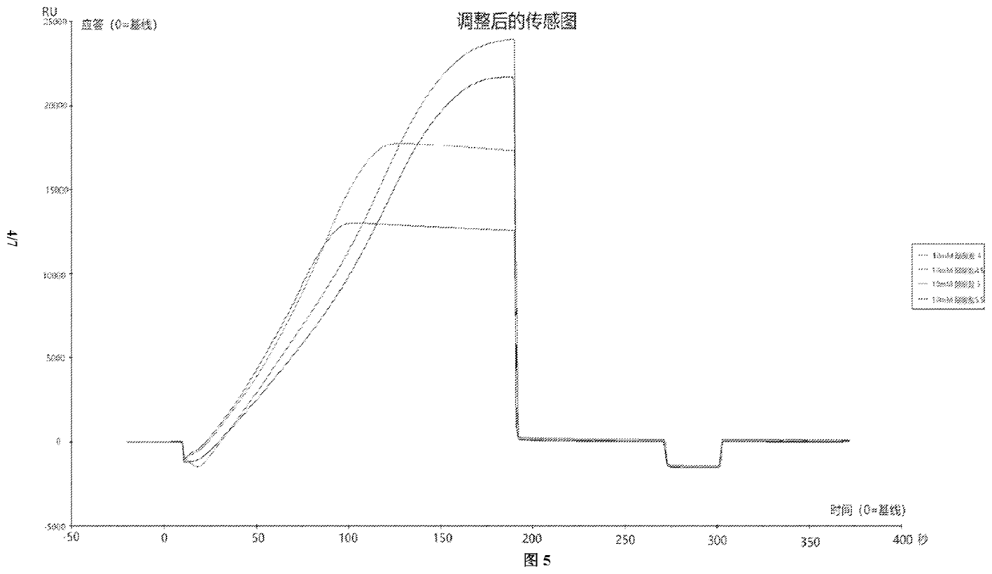

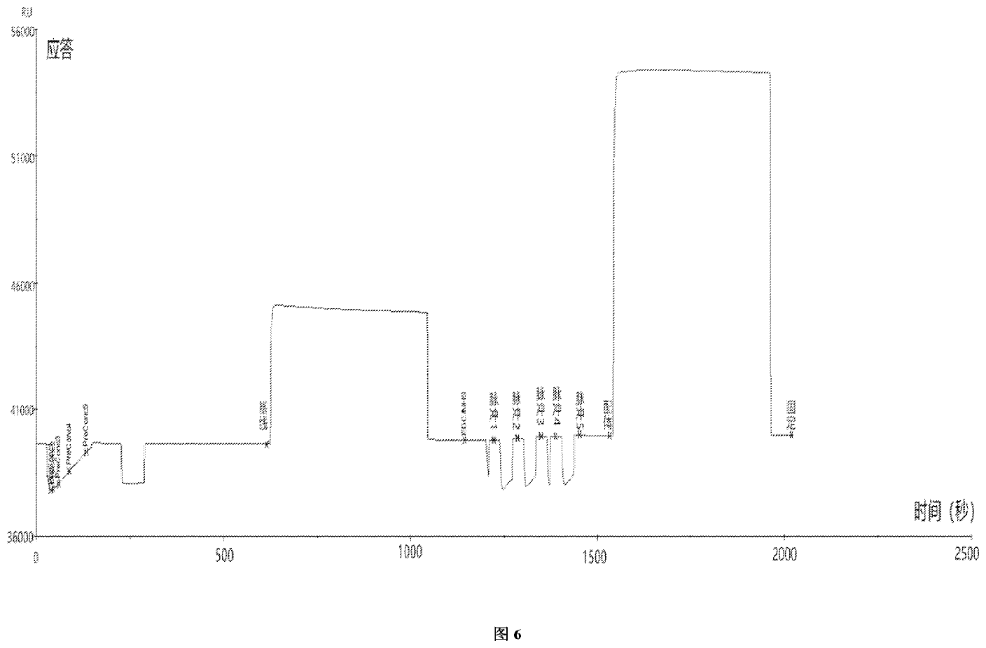

10.1. The CHO-K1 and CHO-K1-BCMA cell strains were thawed from liquid nitrogen, and the cell state was adjusted to log phase.
10.2. The two types of cells were divided into several portions, with 5×10⁵ cells in each portion.
10.3. The expressed single-domain antibodies 48-11 (the amino acid sequence is set forth in SEQ ID NO: 15), D1 (the amino acid sequence is set forth in SEQ ID NO: 16), C6 (the amino acid sequence is set forth in SEQ ID NO: 17), C2 (the amino acid sequence is set forth in SEQ ID NO: 18) and D10 (the amino acid sequence is set forth in SEQ ID NO: 19) were each used to incubate the target cells. They were well mixed and incubated at room temperature for 1 h.
10.4. The mixtures were centrifuged at 800×g at room temperature for 5 min, and the supernatants containing single-domain antibodies were removed. The cells were washed 3 times with PBS.
10.5. 1 μL of PE-labeled Anti-human IgG was added, and they were well mixed and incubated at room temperature in the dark for 30 min.
10.6. The mixtures were centrifuged at 800×g at room temperature for 5 min, and the supernatants containing PE-labeled Anti-human IgG were removed. The cells were washed 3 times with PBS.
10.7. The cells were resuspended in 500 μL of PBS, and the suspensions were assayed by flow cytometry. The results are shown in FIG. 4: the single-domain antibodies 48-11 and D1 have the best ability to bind to the CHO-K1-BCMA recombinant cell strain. The single-domain antibodies were to be purified and assayed for affinity.

### Example 11. Expression and Purification of Single-Domain Antibodies

11.1. The LVTransm transfection reagent and the single-domain antibody expression vector Lenti-hIgG1-Fc2 were taken out of the refrigerator, thawed at room temperature and pipetted up and down until they were each well mixed. PBS buffer was taken out and warmed to room temperature. To a well of a 6-well plate was added 2 mL of PBS followed by 130 μg of Lenti-hIgG1-Fc2. The mixture was pipetted up and down until it was well mixed, and 400 μL of LVTransm was then added. The mixture was immediately pipetted up and down until it was well mixed, and was left to stand at room temperature for 10 min.
11.2. To 50 mL of 293F cells was added the DNA/LVTransm complex, and they were well mixed by gentle shaking. The cells were placed in a 37° C., 5% CO₂ incubator and cultured at 130 RPM for 6-8 h, and 50 mL of fresh FreeStyle™ 293 medium was then added. The cells were placed back into the incubator and cultured again.
11.3. After 7 days of continuous culture, the culture was centrifuged, and the medium supernatant was collected and filtered through a 0.45 μm filter membrane. The filtrate was transferred to a sterile centrifuge tube, and the antibodies were purified through a Protein A column to finally obtain the single-domain antibodies.

### Example 12. Humanization of Single-Domain Antibodies

Antibodies of alpaca origin may cause human-specific immune responses, and therefore the sequences of both the anti-BCMA single-domain antibodies described above were humanized.

The single-domain antibody sequences obtained in Example 11 were aligned with the IMGT database, and the information about the antibody CDRs and FRs, as well as the amino acid position numbers, was obtained according to the IMGT numbering scheme for antibody CDRs. With the human germline DP-47 sequence as a template, the original FR sequences of the antibodies were replaced and the CDR sequences of the antibodies were retained, so that the antibodies were humanized. The humanized sequences of hu4811 and huD1 retained the original FR2 sequences of the antibodies that affect CDR3 conformation. The humanized sequences of hu4811FGLF and huD1FGLF contained replacements for FR1, FR3 and FR4 sequences and contained T45A, E49G and R50L mutations in FR2. To verify the affinity of the humanized single-domain antibodies, the original single-domain antibodies and the humanized single-domain antibodies designed were subjected to fusion protein expression. The affinity of the original single-domain antibodies and the humanized antibodies was measured by Biacore. The obtained humanized antibodies were designated hu4811 (amino acid sequence SEQ ID NO: 22, nucleic acid sequence SEQ ID NO: 28), hu4811FGLF (amino acid sequence SEQ ID NO: 23, nucleic acid sequence SEQ ID NO: 29), huD1 (amino acid sequence SEQ ID NO: 20, nucleic acid sequence SEQ ID NO: 26) and huDIFGLF (amino acid sequence SEQ ID NO: 21, nucleic acid sequence SEQ ID NO: 27).

### Example 13. Assays for Affinity of Single-Domain Antibodies

The BCMA-Fc recombinant protein was immobilized to a CM5 chip with 10 mM acetate buffer, and the ability of each of the above-prepared single-domain antibodies obtained by screening to bind to the BCMA-Fc recombinant protein was measured with each of the single-domain antibodies as a mobile phase.

### 1. Reagent Preparation

Running reagent: containing 10 mM N-(2-hydroxyethyl) piperazine-N-2 sulfonic acid (HEPES), 150 mM sodium chloride (NaCl), 3 mM ethylenediaminetetraacetic acid (EDTA) and 0.005% Tween-20, pH adjusted to 7.4.

A human IgG (Fc) capture kit comprising a mouse anti-human IgG (Fc) antibody, an immobilization reagent (sodium acetate, pH 5.0) and a regeneration reagent (magnesium chloride).

An amino coupling kit comprising N-hydroxysuccinimide (NHS), 1-ethyl-(3-dimethylaminopropyl) carbodiimide hydrochloride (EDC) and ethanolamine (pH 8.5). To each tube of EDC and NHS was added 10 mL of deionized water, and EDC and NHS were aliquoted and stored at −18° C. or lower, with a two-month shelf life.

### 2. Chip Preparation

The mouse anti-human IgG (Fc) antibody was diluted with the immobilization reagent (sodium acetate, pH 5.0): 950 μL of the immobilization reagent was added to 50 μL of the mouse anti-human IgG (Fc) antibody. The dilution was used for immobilization in eight channels. First, the surface of the CM5 chip was activated for 360 s with EDC and NHS at a flow rate of 10 μL/min. Then, the mouse anti-human IgG (Fc) antibody was injected into the channels (channels 1-8, Fc1,2) at a flow rate of 10 μL/min for about 360 s, with the level of immobilization at about 7000 to 14,000 RU. Finally, the chip was blocked with ethanolamine at 10 μL/min for 420 s.

### 3. Buffer Exchange

Buffer exchange was performed for human BCMA protein using a desalting column and the corresponding running reagent, and the concentration of the sample after exchange was determined.

### 4. Ligand Capture

The antibody was diluted to 10 μg/mL with the running reagent, and the dilution was injected into the experimental channels (Fc2) for human IgG (Fc) capture at a flow rate of 10 μL/min at about 300 RU. The reference channels (Fc1) did not require ligand capture.

### 5. Analyte Multicycle Analysis

Human BCMA protein was diluted 2-fold with the running reagent. The diluted human BCMA protein was injected into the experimental channels and the reference channels in sequence at a flow rate of 30 μL/min, and corresponding periods of association and dissociation were allowed. The association and dissociation steps were all performed in the running reagent. After each concentration analysis, the chip needed to be regenerated with magnesium chloride at a flow rate of 20 μL/min for 30 s to wash away the ligand and undissociated analyte. For the next concentration analysis, the experimental channels needed to recapture the same amount of ligand.

### 6. Data Analysis

A KD value was calculated for each sample using Biacore 8K analysis software Biacore Insight Evaluation Software. The reference channels (Fc1) were used for background subtraction.

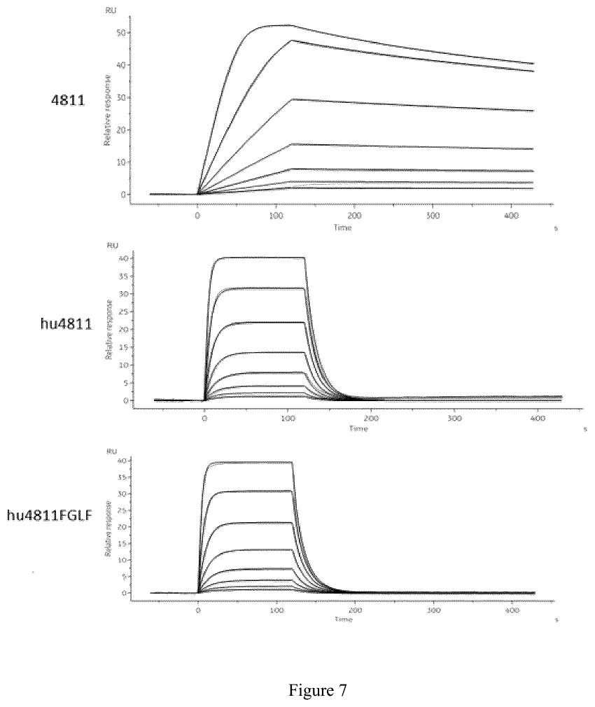

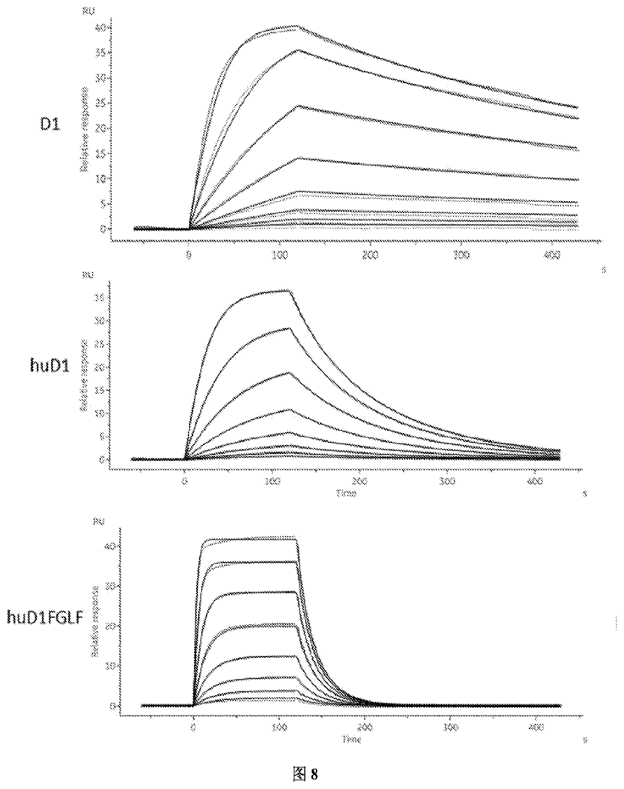

The results are shown in FIG. 7, FIG. 8 and Table 9.

TABLE 9
The results of the binding of the single-domain antibodies to
BCMA-Fc recombinant protein

| | Ka (M⁻¹s⁻¹) | Kd (s⁻¹) | KD (M) |
|---|---|---|---|
| D1 | 8.73E+06 | 2.12E−03 | 2.43E−10 |
| huD1 | 7.41E+06 | 1.41E−02 | 1.90E−09 |
| huD1FGLF | 6.26E+06 | 5.79E−02 | 9.25E−09 |
| 4811 | 3.42E+07 | 1.30E−03 | 3.79E−11 |
| hu4811 | 7.19E+06 | 6.98E−02 | 9.71E−09 |
| hu4811FGLF | 1.24E+07 | 1.26E−01 | 1.02E−08 |

The BCMA single-domain antibodies D1 and 4811, as well as the humanized antibodies hu4811/hu4811FGLF or huD1/huD1FGLF, have high affinity for human BCMA protein.

1. An isolated antigen-binding fragment competing for binding to BCMA protein with a reference antibody, wherein the reference antibody comprises a heavy chain variable region; the heavy chain variable region of the reference antibody may comprise HCDR1, HCDR2 and HCDR3; the HCDR1 comprises amino acid sequences set forth in SEQ ID NO: 2 and SEQ ID NO: 9, the HCDR2 comprises amino acid sequences set forth in SEQ ID NO: 4 and SEQ ID NO: 11, and the HCDR3 comprises amino acid sequences set forth in SEQ ID NO: 6 and SEQ ID NO: 13.

2. The isolated antigen-binding fragment according to claim 1, including single-domain antibodies.

3. The isolated antigen-binding fragment according to claim 1, wherein the isolated antigen-binding fragment comprises a heavy chain variable region comprising HCDR1, HCDR2 and HCDR3, and the HCDR1 comprises an amino acid sequence set forth in any one of SEQ ID NO: 2 and SEQ ID NO: 9.

4. The isolated antigen-binding fragment according to claim 3, wherein the HCDR2 comprises an amino acid sequence set forth in any one of SEQ ID NO: 4 and SEQ ID NO: 11.

5. The isolated antigen-binding fragment according to claim 3, wherein the HCDR3 comprises an amino acid sequence set forth in any one of SEQ ID NO: 6 and SEQ ID NO: 13.

6. The isolated antigen-binding fragment according to claim 1, wherein the isolated antigen-binding fragment comprises a heavy chain variable region comprising H-FR1, H-FR2, H-FR3 and H-FR4, and the H-FR1 comprises an amino acid sequence set forth in SEQ ID NO: 49 or SEQ ID NO: 50.

7. The isolated antigen-binding fragment according to claim 6, wherein the isolated antigen-binding fragment comprises a heavy chain variable region comprising H-FR1, H-FR2, H-FR3 and H-FR4, and the H-FR1 comprises an amino acid sequence set forth in any one of SEQ ID NO: 1, SEQ ID NO: 8 and SEQ ID NO: 30.

8. The isolated antigen-binding fragment according to claim 6, wherein the H-FR2 comprises an amino acid sequence set forth in SEQ ID NO: 51 or SEQ ID NO: 52.

9. The isolated antigen-binding fragment according to claim 8, wherein the H-FR2 comprises an amino acid sequence set forth in any one of SEQ ID NO: 3, SEQ ID NO: 10 and SEQ ID NO: 33.

10. The isolated antigen-binding fragment according to claim 6, wherein the H-FR3 comprises an amino acid sequence set forth in SEQ ID NO: 53 or SEQ ID NO: 54.

11. The isolated antigen-binding fragment according to claim 10, wherein the H-FR3 comprises an amino acid sequence set forth in any one of SEQ ID NO: 5, SEQ ID NO: 12 and SEQ ID NO: 31.

12. The isolated antigen-binding fragment according to claim 6, wherein the H-FR4 comprises an amino acid sequence set forth in SEQ ID NO: 55 or SEQ ID NO: 56.

13. The isolated antigen-binding fragment according to claim 12, wherein the H-FR4 comprises an amino acid sequence set forth in any one of SEQ ID NO: 7, SEQ ID NO: 14 and SEQ ID NO: 32.

14. The isolated antigen-binding fragment according to claim 1, wherein the isolated antigen-binding fragment comprises a heavy chain variable region comprising an amino acid sequence set forth in SEQ ID NO: 47 or SEQ ID NO: 48.

15. The isolated antigen-binding fragment according to claim 1, wherein the isolated antigen-binding fragment comprises a heavy chain variable region comprising amino acid sequences of SEQ ID NO: 15, SEQ ID NO: 16, SEQ ID NO: 20, SEQ ID NO: 21, SEQ ID NO: 22 and SEQ ID NO: 23.

16. An isolated nucleic acid molecule comprising a polynucleotide encoding the isolated antigen-binding fragment according to claim 1.

17. The isolated nucleic acid molecule according to claim 16, comprising one or more polynucleotide sequences selected from the group consisting of SEQ ID NOs: 24-29.

18. A vector comprising the isolated nucleic acid molecule according to claim 16.

19. A host cell comprising the isolated nucleic acid molecule according to claim 16.

20. A method for preparing the isolated antigen-binding fragment according to claim 1, wherein the method comprises culturing the host cell comprising the isolated nucleic acid molecule under such conditions that the isolated antigen-binding fragment is expressed.

21. A pharmaceutical composition comprising the isolated antigen-binding fragment according to claim 1, a nucleic acid molecule comprising the isolated antigen-binding fragment according to claim 1, a vector comprising the isolated antigen-binding fragment according to claim 1 and/or a host cell comprising the isolated antigen-binding fragment according to claim 1, and optionally a pharmaceutically acceptable adjuvant.

22. Use of the isolated antigen-binding fragment according to claim 1, a nucleic acid molecule comprising the isolated antigen-binding fragment according to claim 1, a vector comprising the isolated antigen-binding fragment according to claim 1 and/or a host cell comprising the isolated antigen-binding fragment according to claim 1 in preparing a medicament for preventing or treating a disease or condition.

23. Use of the isolated antigen-binding fragment according to claim 1, a nucleic acid molecule comprising the isolated antigen-binding fragment according to claim 1, a vector comprising the isolated antigen-binding fragment according to claim 1 and/or a host cell comprising the isolated antigen-binding fragment according to claim 1 in preparing a chimeric antigen receptor (CAR) and a chimeric antigen receptor T cell (CART).

24. A host cell comprising the isolated nucleic acid molecule according to claim 18.
